(19) United States
(12) Patent Application Publication
(10) Pub. No.: US 2025/0283226 A1
(43) Pub. Date: Sep. 11, 2025

(54) ELECTROLYSIS SYSTEM FOR HYDROGEN PRODUCTION AND CARBON DIOXIDE CAPTURE AND DELIVERY

(71) Applicant: UCHICAGO ARGONNE, LLC, Chicago, IL (US)

(72) Inventors: YuPo J. LIN, Naperville, IL (US); Thomas LIPPERT, Naperville, IL (US)

(73) Assignee: UCHICAGO ARGONNE, LLC, Chicago, IL (US)

(21) Appl. No.: 19/075,748

(22) Filed: Mar. 10, 2025

Related U.S. Application Data
(60) Provisional application No. 63/562,835, filed on Mar. 8, 2024.

(51) Int. Cl. C25B 1/14; C12M 1/00; C12P 5/02; C25B 1/04; C25B 9/23; C25B 9/77; C25B 11/031; C25B 15/08

(52) U.S. Cl. CPC C25B 1/14; C12M 45/07; C12P 5/023; C25B 1/04; C25B 9/23; C25B 9/77; C25B 11/031; C25B 15/081; C25B 15/083

(57) ABSTRACT

An electrochemical reactor for capturing carbon dioxide and producing bicarbonate and hydrogen is described herein. The electrochemical reactor is useful for, among other things, converting biogas to a bicarbonate and hydrogen feedstock for biomethanation. The reactor comprises at least one reactor unit comprising an electrolyzer cell and at least one alkaline water electrolysis (AWE) cell adjacent to the electrolyzer cell. The electrolyzer cell comprises an anode spaced from a cathode by an ion exchange membrane between the anode and the cathode; and the electrolyzer cell is adapted and arranged to allow a flow of a neutral liquid electrolyte to contact the anode and the cathode. The ion exchange membrane can be a cation exchange membrane (CEM), or an anion exchange membrane (AEM). The AWE cell comprises a second anode spaced from a second cathode by a porous diaphragm.

### CROSS-REFERENCE TO RELATED APPLICATIONS

This application claims the benefit of U.S. provisional Application No. 63/562,835, filed on Mar. 8, 2024, which is incorporated herein by reference in its entirety.

### CONTRACTUAL ORIGIN OF THE INVENTION

The United States Government has rights in this invention pursuant to Contract No. DE-AC02-06CH11357 between the United States Government and UChicago Argonne, LLC representing Argonne National Laboratory.

### FIELD OF THE INVENTION

This invention relates to an electrolysis system for hydrogen production and carbon dioxide capture and delivery.

### BACKGROUND OF THE INVENTION

Environmentally acceptable waste-based energy production, methanol production, and processes rely on efficient capture and removal of carbon dioxide from carbon dioxide-rich gasses such as biologically generated biogas, flue gasses, vehicle emissions, and the like. For example, converting carbon dioxide produced in wastewater treatment to energy is a strategy for ensuring water and energy resilience. Using renewable resources such as solar and wind for carbon dioxide utilization is also an important strategy to reduce the impact of greenhouse gasses on climate change. Biological conversion of organic materials such as food waste, wastewater and waste-treatment sludge can be used on a large scale. For example, carbon dioxide produced in anaerobic digesters and flue gasses can be used as feedstocks for methane production in hydrogenotrophic methanogenesis bioreactors (biomethanation).

Although microbial populations in biomethanation reactors are robust with respect to toxic compounds in the feed streams there are still challenges in producing high-purity methane through biomethanation, including (1) the low aqueous solubility of carbon dioxide and (2) obtaining a suitable, convenient and cost effective source of hydrogen. The low solubility of carbon dioxide leads to low carbon dioxide utilization in bioreactors operated at atmospheric pressure. The source, production cost and storage of hydrogen frequently makes the economics and investment potential of biological methane production unfavorable.

Because of the aforementioned challenges there is an ongoing need for alternative processes for capture and delivery of carbon dioxide, and for hydrogen generation and delivery, for biological production of high-purity methane from organic/biological waste streams. The apparatus and methods described herein address this need.

### SUMMARY OF THE INVENTION

An electrochemical reactor for capturing carbon dioxide and producing a mixed gas stream containing carbon dioxide and hydrogen is described herein. The electrochemical reactor is useful for, among other things, converting CO₂-rich gas streams (e.g., biogas) to bicarbonate/carbon dioxide and hydrogen feedstocks for biomethanation or methanol production. The reactor comprises at least one reactor unit comprising an electrolyzer (E) cell and at least one alkaline water electrolysis (AWE) cell adjacent to, and electrically insulated from, the electrolyzer cell, and is sometimes referred to herein as an E/AWE rector.

The electrolyzer cell comprises a first anode spaced from a first cathode by an ion exchange membrane between the first anode and the first cathode; and the electrolyzer cell is adapted and arranged to allow a flow of a first liquid electrolyte to contact the first anode and the first cathode. The ion exchange membrane can be a cation exchange membrane (CEM), or an anion exchange membrane (AEM). The AWE cell comprises a second anode spaced from a second cathode by a porous diaphragm, and the AWE cell is adapted and arranged to allow a flow of a second liquid electrolyte to contact the second anode and the second cathode. The electrolyzer cell is electrically insulated from the AWE cell by a first electrical insulator between the AWE cell and the electrolyzer cell. Second and third electrical insulators are situated adjacent to the sides of the electrolyzer cell and AWE cell that are opposite the first electrical insulator. In some embodiments, the reactor unit comprises multiple AWE cells (e.g., two to ten AWE cells) in a stack, which are insulated from each other by additional electrical insulators flanking each AWE cell. The anodes and cathodes typically are in the form of porous or non-porous metal plates. In the case of porous electrodes (anodes and cathodes) with a continuous pore structure, electrolyte can contact the electrodes by flowing through the pore system of the electrodes. In the case of non-porous electrodes, enough space between the electrodes and adjacent insulators is provided to allow the electrolyte to contact the electrodes by flowing over/across the non-porous surfaces of the electrodes.

In some embodiments, each reactor unit includes two to eleven (e.g., seven) AWE cells per electrolyzer cell. In such embodiments, a manifold can be utilized to distribute the flow of the second electrolyte to each anode and cathode. Similarly, separate manifolds can be utilized to collect and combine effluents from the anodes of the AWE cells, and to collect and combine effluents from the cathodes of the AWE cells, respectively.

In some embodiments, the electrolyzer cell includes a first inlet adapted and arranged to allow a flow of a first liquid electrolyte to contact the first anode; a first outlet positioned opposite the first inlet in fluid communication with the first inlet; a second inlet adapted and arranged to allow a flow of the first liquid electrolyte to contact first cathode; and a second outlet positioned opposite the second inlet in fluid communication with second inlet. The AWE cell includes a third inlet adapted and arranged to allow a flow of a second liquid electrolyte to contact the second anode; a third outlet positioned opposite the third inlet in fluid communication with the third inlet; a fourth inlet adapted and arranged to allow a flow of the second liquid electrolyte to contact the second cathode; and a fourth outlet positioned opposite the fourth inlet in fluid communication with the fourth inlet. The first inlet and first outlet together direct the flow of the first liquid electrolyte (e.g., an aqueous solution of a neutral salt such as sodium sulfate) to contact the anode of the electrolyzer cell; and the second inlet and second outlet together direct the flow of the first liquid electrolyte to contact the cathode of the electrolyzer cell. Similarly, the third inlet and third outlet together direct the flow of the second liquid electrolyte (e.g., a basic aqueous solution such as aqueous sodium hydroxide) to contact the anode of the AWE; and the fourth inlet and fourth outlet together direct the flow of the second liquid electrolyte to contact the cathode of the AWE.

In use, a flow of the first liquid electrolyte, which comprises a neutral salt (e.g., aqueous sodium sulfate), contacts the anode and cathode of the electrolyzer cell, e.g., via the first and second inlets and outlets; and a flow of the second liquid electrolyte, which is alkaline (e.g., aqueous potassium hydroxide or sodium hydroxide), contacts the anode and cathode of the AWE cell, e.g., via the third and fourth inlets and outlets. At the same time, an electrical potential is applied across the anode and cathode of the electrolyzer cell, and the anode and cathode of the AWE cell (i.e., by placing the anode and cathode of each cell in circuit with a direct current (DC) power source). The voltage across the anode and cathode of the electrolyzer cell generates oxygen at the anode and hydrogen at the cathode, and simultaneously generates hydrogen ion (acid) in the first liquid electrolyte flowing over or through the anode and generates hydroxide ion (alkali) in the first liquid electrolyte flowing over or through the cathode. The net charges in the electrolytes contacting the anode and the cathode of the electrolyzer cell are balanced by a directed flow of ions across the ion exchange membrane. Similarly, the voltage across the anode and cathode of the AWE cell generates oxygen at the anode and hydrogen at the cathode, and simultaneously generates hydrogen ion in the second liquid electrolyte flowing through the anode and generates hydroxide ion in the electrolyte solution flowing through the cathode of the AWE cell. Typically, the concentration of base utilized in the second electrolyte is high enough so that hydrogen ions generated at the anode and hydroxide ions generated at the cathode of the AWE cells do not significantly affect the pH of the second electrolyte.

The alkali generated at the cathode of the electrolyzer cell can be used to capture carbon dioxide as bicarbonate from biogas or other carbon dioxide-rich gasses, while the acid generated at the anode of the electrolyzer cell can be used to neutralize or partially neutralize the bicarbonate to regenerate purified carbon dioxide gas, e.g., for use as a carbon feedstock for biomethanation or other CO₂ reduction reactor. In addition, hydrogen generated at the cathodes of the electrolyzer and AWE cells can be used as the hydrogen feedstock, e.g., for biomethanation.

The apparatus and system described herein provides and improvement on electrochemical technology designed to produce hydrogen from aqueous solution while simultaneously capturing carbon dioxide from biogas (65% methane and 35% carbon dioxide) and turning it into aqueous bicarbonate. The produced hydrogen can be supplied to a downstream biomethanation reactor along with the captured carbon dioxide for microbiological conversion to methane. Thanks to the carbon dioxide capture, the biogas stream is purified and reaches high methane concentrations (>95%) so that the gaseous effluents from both the electrochemical reactor and the biomethanation reactor can be supplied to the gas grid as renewable natural gas (RNG).

Previously described technology was challenged by high pH levels (>10) in the liquid carrying the dissolved carbon dioxide, thus retarding the activity of the hydrogenotrophic methanogens in the biomethanation reactor. Furthermore, it was a challenge for the previously described reactors to produce enough hydrogen to meet the stoichiometry of the biomethanation reaction of 4 moles of hydrogen per each mole of carbon dioxide captured. Reactors described in co-owned U.S. Provisional Application No. 63/448,417 and non-provisional application Ser. No. 18/586,922 (collectively referred to as “USPA '417”), which are sometimes referred to as ECC/AWE reactors, addressed these limitations by adding an acid producing chamber in the electrochemical reactor. Thanks to this acid stream, the pH of the carbon dioxide-containing capture stream could be corrected to near neutral levels, thus allowing for an uninhibited biomethanation process. Inclusion of additional cells for alkaline water electrolysis (AWE) also enabled stoichiometric delivery of hydrogen and carbon dioxide. While these modifications successfully addressed the challenges encountered in previous systems, further challenges have been identified, including:
Inclusion of bipolar membranes (BPMs) in the ECC/AWE reactor increases the voltage drop and energy consumption of the reactor compared to simple alkaline hydrolysis.
Current state-of-the-art BPMs are typically limited to current densities between 50 mA/cm² and 100 mA/cm², which is significantly lower than typical current densities used for full-scale AWE (i.e., 1,000 mA/cm²), thus challenging the compatibility of both technologies.
Over time, BPMs can show increased voltage drops due to delamination caused by high current densities (100 mA/cm²) and intrusion of carbon dioxide gas into the bipolar junction.

The E/AWE apparatus described herein addresses those limitations using a cell design that does not include bipolar membranes, but still retains the ability to capture carbon dioxide and generate hydrogen, as described herein below. This affords the advantage of a lower voltage drop across the cell and eliminates the challenges of using BPMs (i.e., low maximum current densities, and system performance degradation due to BPM delamination). In addition, a prototype of an E/AWE reactor described herein exhibited a surprising 67% percent reduction in energy consumption compared to the ECC/AWE reactor of USPA '417. The E/AWE reactor described herein is useful for any downstream carbon dioxide utilization system, e.g., in conjunction with an electrochemical catalytic reactor, a bioreactor for methane production, or for supplying carbon dioxide and acidic feedstocks to algae ponds.

In some embodiments, a specified voltage is applied across the anode and cathode of the electrolyzer cell while a stream of an aqueous solution of a neutral salt (the first liquid electrolyte; e.g., aqueous sodium sulfate) is contacting the anode and the cathode. Hydrogen gas and hydroxide ions are electrochemically generated at the cathode, converting the neutral solution to an alkaline solution, which passes out of the electrolyzer cell where the hydrogen gas is separated from the alkaline solution. A stream of biogas (methane and carbon dioxide) generated in a bioreactor is contacted with the resulting hydrogen-depleted alkaline solution to capture carbon dioxide from the biogas as aqueous bicarbonate. At the same time, oxygen gas and hydrogen ions are electrochemically generated at the anode of the electrolyzer cell, converting the neutral salt solution to an acidic solution, which passes out of the electrolyzer cell, where the oxygen gas is separated from the acidic solution.

In some embodiments, the first liquid electrolyte (e.g., aqueous sodium sulfate) feeding the electrolyzer cell is generated by bringing together the bicarbonate solution generated by contacting the alkaline effluent with a carbon dioxide-rich gas, such as biogas, with the acidic solution generated at the anode of the electrolyzer cell. This liberates gaseous carbon dioxide and generates an aqueous neutral salt solution (e.g., aqueous sodium sulfate), which is utilized as the electrolyte for the electrolyzer cell. In some embodiments, the resulting recombined solution is sparged with the produced hydrogen in a stripping column. This enhances liquid/gas mass transfer and lowers the partial pressure of carbon dioxide, thus promoting a faster release of carbon dioxide from the liquid phase.

The carbon dioxide gas recovered from neutralization of the bicarbonate solution and hydrogen generated at the cathode of the electrolyzer cell can be used as a carbon and hydrogen feedstocks for a biomethanation reactor to produce methane. The hydrogen gas from the electrolyzer cell is supplemented with hydrogen gas recovered from the AWE cell(s) to adjust the ratio of carbon dioxide to hydrogen to an optimal level for biomethanation. Another example of utilizing the CO₂ and H₂ is the gas-water shift reaction to produce CO (i.e., CO₂+H₂=CO+H₂O).

The number of AWE cells in each reactor unit is chosen, in conjunction with the size and capacity of the electrolyzer cell, to generate the optimum stoichiometry of hydrogen to carbon dioxide for biological methane production (e.g., 4 moles of hydrogen gas to 1 mole of carbon dioxide for hydrogenotrophic methanogenesis bioreactors). In some embodiments, the electrochemical reactor comprises multiple reactor units working in tandem so as to efficiently handle the amount of biogas feeding the electrochemical reactor from the anaerobic digester. Methane recovered from the biogas that passes through the second gas/liquid separator is recovered, thereby boosting the overall yield of methane from the system.

The apparatus described herein, which does not require bipolar membranes, provides a number of advantages when used for biological methane production, including the ability to adjust the ratio of the hydrogen and carbon dioxide feedstocks for optimum biomethanation without using a separate hydrogen gas source. The system generates high-purity renewable methane, at a lower energy consumption for operation of the apparatus compared to a system using bipolar membranes, while retaining the ability to tailor the ratio of carbon dioxide to hydrogen for optimal biomethanation.

In addition, commercial algae ponds often require enhancement with carbon dioxide or a carbon dioxide source (e.g., bicarbonate) to promote efficient algae growth. In addition, the pH of algae bonds often needs to be adjusted during pond operation. When used in conjunction with algae ponds, the E/AWE reactor described herein can supply needed carbon dioxide in the form of bicarbonate solution or as carbon dioxide gas, and acid can be used for pH adjustment, when needed.

Also described herein is a neutral water electrolysis reactor system utilizing the electrolyzer cell portion of the E/AWE system described herein without the AWE cells, to obtain renewable natural gas from, e.g., biogas sources. Power-to-gas approaches that convert excess renewable electricity into green hydrogen gas (H₂) or renewable natural gas (RNG) represent promising energy storage technologies due to their long-term gas storability, versatility, and low carbon footprint. A new power-to-gas approach is presented herein, which couples H₂ and RNG production from biogas in a unit operation process via neutral water electrolysis (NWE). The process leverages the inherent capability of the water electrolysis reaction to produce acids and bases from a pH-neutral electrolyte that can then serve as agents for pH-mediated carbon capture and release. Results demonstrate that NWE features the same H₂ production rates and purities as a reference alkaline water electrolysis (AWE) cell, while simultaneously facilitating near-complete CO₂ capture (>99%) from a synthetic biogas stream (65% CH4, 35% CO₂) with resulting maximum CH₄ qualities of >99%, suitable for direct natural gas grid injection. With an added energy consumption as low as 1 kWh per kg CO₂ captured and released, chemical-free operation, the same footprint as an AWE cell, and a reactor made exclusively from commercially available materials, the presented NWE approach provides a novel, sustainable power-to-gas technology with high market adoption potential.

This approach improves the viability of power-to-gas technology by integrating H₂ and RNG production within a single unit operation process, targeting reduced investment costs, operational complexity, and equipment footprint. Operation of an electrolysis cell with a pH-neutral electrolyte (e.g., aqueous Na₂SO₄) instead of a highly alkaline electrolyte (e.g. KOH for AWE) avoids overpowering the electrolyte with hydroxide anions. Thus, the NEW approach can utilize the inherent capability of the water electrolysis reaction to create an appreciable pH gradient between anolyte and catholyte, thus enabling coupling of H₂ production with pH-mediated CO₂ capture and release:

$$Cathode reaction : 4 {H}_{2} O + 4 {e}^{- -} \to 2 {H}_{2} + 4 {OH}^{-} \qquad ( Eq . 1 )$$

$$Anode reaction : 2 {H}_{2} O \to {O}_{2} + 4 {H}^{+} + 4 {e}^{- -} \qquad ( Eq . 2 )$$

Continuing with the example of Na₂SO₄ electrolyte, the NWE cell produces sodium hydroxide at the cathode (Na⁺+OH⁻⁻→NaOH) and sulfuric acid at the anode (SO4₂⁻⁻+2 H⁺→H₂SO₄). The alkaline catholyte can then be used as a CO₂ capture agent (e.g., NaOH+CO₂→NaHCO₃), while the acidic anolyte facilitates subsequent CO₂ release and electrolyte regeneration (e.g., 2 NaHCO₃+H₂SO₄→Na₂SO₄+2 CO₂+2 H₂O). The process comes at the cost of an increased voltage drop due to the lower electrolyte conductivity (as compared to highly concentrated KOH common in AWE systems) and the added Nernstian potential from the created pH gradient (i.e., 0.059·ΔpH [V]). However, it also benefits from a simple two-compartment cell design (same footprint as AWE) and eliminates the need for costly, easy-to-deteriorate, and current-limiting bipolar membranes (as used in other electrochemical CO₂ capture technologies like electrodialysis or electrodeionization). 12,13

The following non-limiting embodiments are provided to describe certain aspects and features of the electrochemical reactors and methods described herein.

Embodiment 1 is an electrochemical reactor useful for capturing carbon dioxide and generating hydrogen; the reactor comprising: at least one reactor unit including an electrolyzer cell and a first alkaline water electrolysis (AWE) cell electrically insulated from the electrolyzer cell, the reactor unit being flanked by electrical insulators;
wherein the electrolyzer cell comprises:
(a) a first anode;
(b) a first cathode; and
(c) an ion exchange membrane between the first anode and the first cathode; wherein the ion exchange membrane is selected from the group consisting of a cation exchange membrane and an anion exchange membrane; and
the first AWE cell comprises:
(d) a second anode;
(e) a second cathode; and
(f) a porous diaphragm between the second anode and the second cathode.

Embodiment 2 is the electrochemical reactor of embodiment 1; further comprising:
(i) a first inlet adapted and arranged to allow a flow of a first liquid electrolyte to contact the first anode during use;
(ii) a first outlet positioned opposite the first inlet in fluid communication with the first inlet;
(iii) a second inlet adapted and arranged to allow a flow of the first liquid electrolyte to contact first cathode during use;
(iv) a second outlet positioned opposite the second inlet in fluid communication with second inlet;
(v) a third inlet adapted and arranged to allow a flow of a second liquid electrolyte to contact the second anode during use;
(vi) a third outlet positioned opposite the third inlet in fluid communication with the third inlet;
(vii) a fourth inlet adapted and arranged to allow a flow of the second liquid electrolyte to contact the second cathode during use; and
(viii) a fourth outlet positioned opposite the fourth inlet in fluid communication with the fourth inlet.

Embodiment 3 is the electrochemical reactor of embodiment 1 or embodiment 2, wherein the electrochemical reactor comprises a stack of 2 to about 50 reactor units.

Embodiment 4 is the electrochemical reactor any one of embodiments 1 to 3, wherein each reactor unit comprises 1 to 10 additional AWE cells stacked with the first AWE cell and electrically insulated from the first AWE cell and each other.

Embodiment 5 is the electrochemical reactor of any one of embodiments 2 to 4, further comprising:
a first manifold connecting the third and fourth inlets of each AWE cell to distribute the flow of the second electrolyte to the second anode and second cathode during use;
a second manifold connecting the third outlets of each AWE cell to combine the flow of the second electrolyte exiting each third outlet during use; and
a third manifold connecting the fourth outlets of each AWE cell to combine the flow of the second electrolyte exiting each fourth outlet during use.

Embodiment 6 is the electrochemical reactor of any one of embodiments 1 to 5, wherein the first and second cathodes and the first and second anodes comprise non-porous conductive plates.

Embodiment 7 is the electrochemical reactor of embodiment 6, wherein the non-porous conductive plates comprise a material selected from the group consisting of nickel, titanium, stainless steel, and a Magnéli phase titanium oxide.

Embodiment 8 is the electrochemical reactor of any one of embodiments 1 to 5, wherein the first and second cathodes and the first and second anodes comprise porous conductive plates.

Embodiment 9 is the electrochemical reactor of embodiment 8, wherein the porous conductive plates are porous plates of a material selected from the group consisting of nickel, titanium, stainless steel, and a Magnéli phase titanium oxide.

Embodiment 10 is a method for simultaneously electrochemically generating hydrogen, oxygen, an acidic solution, and a bicarbonate solution comprising the steps of:
(a) providing the electrochemical reactor of any one of embodiments 1 to 9;
(b) contacting a flow of a first liquid electrolyte comprising an aqueous solution of a neutral salt with the first anode and the first cathode of the electrolyzer cell;
(c) contacting a flow of a second liquid electrolyte comprising an aqueous alkaline solution with second anode and the second cathode of the AWE cell;
(d) placing the first anode and the first cathode of the electrolyzer cell in circuit with a DC power source; and
(e) placing the second anode and the second cathode of the AWE cell in circuit with a DC power source;
wherein hydrogen gas and hydroxide ions are electrochemically generated at the first cathode within the flow of the first liquid electrolyte contacting the first cathode, and the flow of the first liquid electrolyte becomes an alkaline first effluent containing hydrogen gas after contact with the first cathode ceases; oxygen gas and hydrogen ions are electrochemically generated at the first anode within the flow of the first liquid electrolyte contacting the first anode, and the flow of the first liquid electrolyte contacting the first anode becomes an acidic second effluent containing oxygen after contact with the first anode ceases; hydrogen gas and hydroxide ions are electrochemically generated at the second cathode within the flow of the second liquid electrolyte contacting the second cathode, and the flow of the second liquid electrolyte becomes an alkaline third effluent containing hydrogen gas after contact with the second cathode ceases; oxygen gas and hydrogen ions are electrochemically generated at the second anode within the flow of the second liquid electrolyte contacting the second anode, and the flow of the second liquid electrolyte contacting the second anode becomes an alkaline fourth effluent containing oxygen after contact with the first anode ceases;
(f) separating and collecting the hydrogen gas from the first effluent to afford a hydrogen-depleted alkaline first effluent;
(g) separating the oxygen gas from the second effluent to afford an oxygen-depleted acidic second effluent;
(h) separating and collecting the hydrogen gas from the third effluent to afford a hydrogen-depleted alkaline third effluent;
(i) separating the oxygen gas from the fourth effluent to afford an oxygen-depleted alkaline fourth effluent;
(j) contacting the hydrogen-depleted alkaline first effluent with a carbon dioxide-rich gas (e.g., biogas, flue gas, etc.) to generate a bicarbonate containing solution by reaction of hydroxide ions in the hydrogen-depleted alkaline first effluent with carbon dioxide in the carbon dioxide-rich gas;
(k) separating and collecting methane from the bicarbonate-containing solution.

Embodiment 11 is the method of embodiment 10, wherein the first liquid electrolyte comprises aqueous sodium sulfate.

Embodiment 12 is the method of embodiment 11, wherein the sodium sulfate is present in the first liquid electrolyte at a concentration of about 5 to about 30 percent by weight (wt %) (e.g., about 15 to about 20 wt %).

Embodiment 13 is the method of any one of embodiments 10 to 12, wherein the second liquid electrolyte comprises aqueous sodium hydroxide or aqueous potassium hydroxide.

Embodiment 14 is the method of embodiment 13, wherein the sodium hydroxide or the potassium hydroxide is present in the second liquid electrolyte at a concentration of about 20 wt % to about 40 wt % (e.g., about 30 to 35 wt %).

Embodiment 15 is the method of any one of embodiments 10 to 14, further comprising the steps of:
(A) combining the hydrogen collected in steps (f) and (h);
(B) contacting the hydrogen-depleted alkaline second effluent with the hydrogen-depleted acidic first effluent to generate carbon dioxide gas and a neutral salt solution; and
(C) separating and collecting the carbon dioxide gas generated in step (B) (e.g., by sparging the resulting combined solution with the produced hydrogen to aid in releasing the carbon dioxide, producing a hydrogen/carbon dioxide mixture).

Embodiment 16 is the method of embodiment 15, further comprising the steps of:
(D) adding the hydrogen from step (A) and the carbon dioxide gas from step (C) to a biomethanation reactor charged with microorganisms for converting hydrogen and carbon dioxide to methane; and
(E) collecting methane generated by the microorganisms.

Embodiment 17 is the method of embodiment 16, further comprising combining the methane collected in step (k) with the methane collected in step (E), e.g., for use as a fuel.

Embodiment 18 is a neutral water electrolysis reactor comprising:
(1) an electrolyzer cell comprising an anode; a cathode; and an ion exchange membrane between the anode and the cathode, separating the electrolyzer cell into an anode chamber and a cathode chamber; wherein the ion exchange membrane is selected from the group consisting of a cation exchange membrane and an anion exchange membrane;
(2) an electrolyte storage tank for supplying an aqueous neutral electrolyte to the electrolyzer cell;
(3) the anode chamber and the cathode chamber being adapted and arranged in fluid communication with the electrolyte storage tank to allow separate flows of the aqueous neutral electrolyte to contact the anode and the cathode, respectively, during use;
(4) a first gas/liquid separator in fluid communication with the anode chamber; the first gas/liquid separator being adapted and arranged to separate gases from an acidic anolyte discharging from the anode chamber during use;
(5) a second gas/liquid separator in fluid communication with the cathode chamber; the second gas/liquid separator being adapted and arranged to separate gases from a basic catholyte discharging from the cathode chamber during use;
(6) the electrolyte storage tank being in fluid communication with the first gas/liquid separator such that a liquid portion of the acidic anolyte discharging from the anode chamber during use is directed into the electrolyte storage tank; and
(7) a porous gas/liquid contactor in fluid communication with the second gas/liquid separator; the second gas/liquid separator being adapted and arranged to direct a flow of a liquid portion of the basic catholyte into the gas/liquid contactor to contact a counterflow of a gas stream comprising carbon dioxide and methane within the gas/liquid contactor during use; the porous gas/liquid contactor also being in fluid communication with the electrolyte storage tank such that basic catholyte containing carbonate and/or bicarbonate ion discharging from the contactor during use is directed into the electrolyte storage tank;
wherein in use, a voltage is applied across the anode and the cathode, and neutral electrolyte from the electrolyte storage tank is circulated through the electrolyzer cell; the gas stream comprising carbon dioxide and methane is introduced into the gas/liquid contactor; water in the neutral electrolyte circulating through the anode chamber is oxidized to form oxygen gas and hydrogen ion, thereby producing the acidic anolyte; water in the neutral electrolyte flowing through the cathode chamber is reduced to form hydrogen gas and hydroxide ion; thereby producing the basic catholyte; the acidic anolyte and basic catholyte containing carbonate and/or bicarbonate entering the electrolyte storage tank regenerate the neutral electrolyte within the tank; the hydrogen gas separated from the basic catholyte in the second gas/liquid separator is collected for later use; the basic catholyte flowing into the gas/liquid contactor reacts with gaseous carbon dioxide from the gas stream to sequester the carbon dioxide as carbonate and/or bicarbonate ions; and a carbon-dioxide-depleted methane-containing gas stream is vented from the gas/liquid contactor and is collected for later use.

Embodiment 19 is the neutral water electrolysis reactor of embodiment 18, wherein the first and second cathodes and the first and second anodes comprise non-porous conductive plates.

Embodiment 20 is the neutral water electrolysis reactor of embodiment 19, wherein the non-porous conductive plates comprise a material selected from the group consisting of nickel, titanium, stainless steel, and a Magnéli phase titanium oxide.

Embodiment 21 is the neutral water electrolysis reactor of embodiment 18, wherein the first and second cathodes and the first and second anodes comprise porous conductive plates.

Embodiment 22 is the neutral water electrolysis reactor of embodiment 21, wherein the porous conductive plates are porous plates of a material selected from the group consisting of nickel, titanium, stainless steel, and a Magnéli phase titanium oxide.

Embodiment 23 is the is the neutral water electrolysis reactor of any one of embodiments 18 to 22 wherein the ion-exchange membrane is a cation-exchange membrane.

The systems, electrochemical reactors, and methods described herein comprise certain novel features and a combination of parts hereinafter fully described, which are illustrated in the accompanying drawings, and particularly pointed out in the appended claims. It is to be understood that various changes in the details may be made without departing from the spirit, or sacrificing any of the advantages of the systems, electrochemical reactors, and methods described herein.

### BRIEF DESCRIPTION OF THE DRAWINGS

FIG. 1 provides a schematic illustration of (A) an exploded view of an electrolyzer cell comprising a cation exchange membrane; and (B) an exploded view of an electrolyzer cell comprising an anion exchange membrane.

FIG. 2 provides a schematic illustration of an electrochemical reactor unit comprising an electrolyzer cell and an AWE cell.

FIG. 3 provides a schematic illustration of an electrochemical reactor unit comprising an electrolyzer cell and seven AWE cells.

FIG. 4 provides a schematic illustration of a partially exploded view of an electrochemical reactor unit comprising an electrolyzer cell and seven AWE cells, in use for generating hydrogen, an acidic effluent comprising sulfuric acid, and an alkaline effluent comprising sodium hydroxide.

FIG. 5 provides a schematic illustration of an electrochemical reactor unit comprising an electrolyzer cell and seven AWE cells integrated into a system for isolating the hydrogen produced by the reactor, isolating the acidic effluent, and generating bicarbonate from biogas contacted with the alkaline effluent.

FIG. 6 provides a partial schematic illustration of the electrochemical reactor unit of FIG. 5 further illustrating conversion of the bicarbonate back to carbon dioxide and simultaneously generating an aqueous sodium sulfate, which is utilized as the first electrolyte for the reactor.

FIG. 7 provides a bar graph comparing the rate of hydrogen generation for E/AWE reactor described herein compared to the rate of an AWE cell.

FIG. 8 provides plots of energy consumption over time for an E/AWE reactor as described herein versus an ECC/AWE reactor.

FIG. 9 schematically illustrates an embodiment of an electrolyzer cell in partially-exploded view (a); and sealed within a housing to provide separate anode and cathode chambers within the electrolyzer cell (b).

FIG. 10 schematically illustrates a neutral water electrolysis reactor system as described herein.

FIG. 11 provides H₂ gas composition (a) and H₂ production rates (b) for both alkaline water electrolysis (AWE) and neutral water electrolysis (NWE) at current densities of 50 and 100 mA/cm². The left two columns show measurement values over time (5 hour experiment for AWE, 15 hour experiment for NWE), while biogas flow rates and corresponding current utilizations (CUs) were increased stepwise every 3 hours in the NWE experiment (from 0.2 to 1.0 mole CO₂ per mole electrons). The right column or (b) shows average values of H₂ production rates over the entire experimental durations, with error bars denoting the standard deviation of the mean.

FIG. 12 provides plots of compositions of upgraded biogas at current densities of 50 mA/cm² (a) and 100 mA/cm² (b) in both once-through and recirculation operation normalized to CH₄ and CO₂, as well as corresponding CO₂ capture rates (c).

### DETAILED DESCRIPTION OF THE PRESENT INVENTION

An electrochemical reactor for capturing carbon dioxide and producing bicarbonate and hydrogen is described herein. The electrochemical reactor is useful for, among other things, converting carbon dioxide-rich gasses, such as biogas, flue gasses, etc., to bicarbonate (and also to purified carbon dioxide) and simultaneously producing hydrogen, all of which can be used as feedstocks for a variety of applications, including, e.g., biomethanation. The reactor comprises at least one reactor unit comprising an electrolyzer cell and at least one alkaline water electrolysis (AWE) cell adjacent to the electrolyzer cell. The electrolyzer cell comprises a first anode spaced from a first cathode by an ion exchange membrane between the first anode and the first cathode; and the electrolyzer cell is adapted and arranged to allow a flow of a first liquid electrolyte to contact the first anode and the first cathode. The ion exchange membrane (IEM) can be a cation exchange membrane (CEM), or an anion exchange membrane (AEM). The AWE cell comprises a second anode spaced from a second cathode by a porous diaphragm.

In use, a flow of a first liquid electrolyte, which comprises a neutral salt (e.g., aqueous sodium sulfate), contacts the anode and cathode of the electrolyzer cell; and a flow of the second liquid electrolyte, which is alkaline (e.g., aqueous potassium hydroxide), contacts the anode and cathode of the AWE cell. At the same time, an electrical potential is applied across the anode and cathode of the electrolyzer cell and the anode and cathode of the AWE cell (i.e., by placing the anode and cathode of each cell in circuit with a DC power source). The voltage across the anode and cathode of the electrolyzer cell generates oxygen at the anode and hydrogen at the cathode, and simultaneously generates hydrogen ion (acid) in the first liquid electrolyte contacting the anode and generates hydroxide ion (alkali) in the first liquid electrolyte contacting the cathode. Similarly, the voltage across the anode and cathode of the AWE cell generates oxygen at the anode and hydrogen at the cathode, and simultaneously generates hydrogen ion in the second liquid electrolyte contacting the anode and generates hydroxide ion in the electrolyte solution contacting the cathode of the AWE cell. The net charges in the electrolytes flowing over or through the anode and the cathode of the electrolyzer cell are balanced by a directed flow of ions across the ion exchange membrane (i.e., a CEM or AEM). Typically, the electric potential applied across the anodes and cathodes of the E/AWE reactor is at least 1.23 Volt or greater (e.g., about 1.23 to about 5 Volts) per reactor cell for a desired current.

Renewable methane can be produced by a number of methods. Anaerobic digestion of organic materials (e.g., wastewater, sludge, food waste, etc.) affords a mixture of carbon dioxide and methane commonly known as biogas. The methane must be separated from the carbon dioxide and purified before it can be used as a fuel. Hydrogenotrophic methanogenesis converts carbon dioxide and hydrogen into methane—a process commonly referred to as biomethanation. The E/AWE reactors described herein can be used to purify methane from biogas and capture the carbon dioxide from the biogas for use as a feedstock for biomethanation. The E/AWE reactor electrochemically generates hydrogen and an alkaline effluent comprising a base such as sodium hydroxide. The hydrogen produced by the E/AWE reactor can be directly used as the hydrogen feedstock for biomethanation. The alkaline effluent from the E/AWE reactor can be used to capture carbon dioxide from biogas as bicarbonate, and liberate relatively pure methane from the biogas. The bicarbonate can then be used to regenerate carbon dioxide for biomethanation. The balance of hydrogen and bicarbonate produced by the E/AWE reactor can be controlled, as described above, to afford a ratio of hydrogen to bicarbonate that is optimal for biomethanation (i.e., a four to one molar ratio of hydrogen to carbon dioxide).

Contact between the alkaline effluent from the E/AWE reactor and the biogas can be facilitated by a gas/liquid contactor, which passively mixes the gas and effluent together for efficient reaction of carbon dioxide with base (e.g., sodium hydroxide) in the alkaline effluent. The gas/liquid contactor typically is a cylindrical vessel containing porous packing material, e.g., small saddle-shaped objects, small rings with a lattice structure, or small spherical objects. When operated in counter-current mode, liquid is trickled from the top and flows to the bottom of the gas/liquid contactor, while the gas phase flows from the bottom to the top.

Anodes and cathodes of the E/AWE reactors described herein typically will comprise an electrically conductive plate, such as titanium, nickel, stainless steel, bismuth, and/or Magnéli phase titanium oxides, as well as electrodes with a rare earth metal coating. The electrically conductive plate can be porous or non-porous. Typically, AWE cells can utilize nickel electrodes, while NWE preferably use rare earth metal-coated anodes. In some embodiments, the anode has a titanium core and an iridium ruthenium coating. The cathode of the NEW cell, which operates under alkaline conditions, can utilize the same materials as the cathodes used in the AWE cells.

The electrical insulators for the E/AWE reactors typically comprise fluoropolymer elastomers, rubber-like polymers, and/or plastics.

The diaphragms of the AWE cell portion of the E/AWE reactors described herein typically comprise a composite separator material made from polysulfone and ZrO₂.

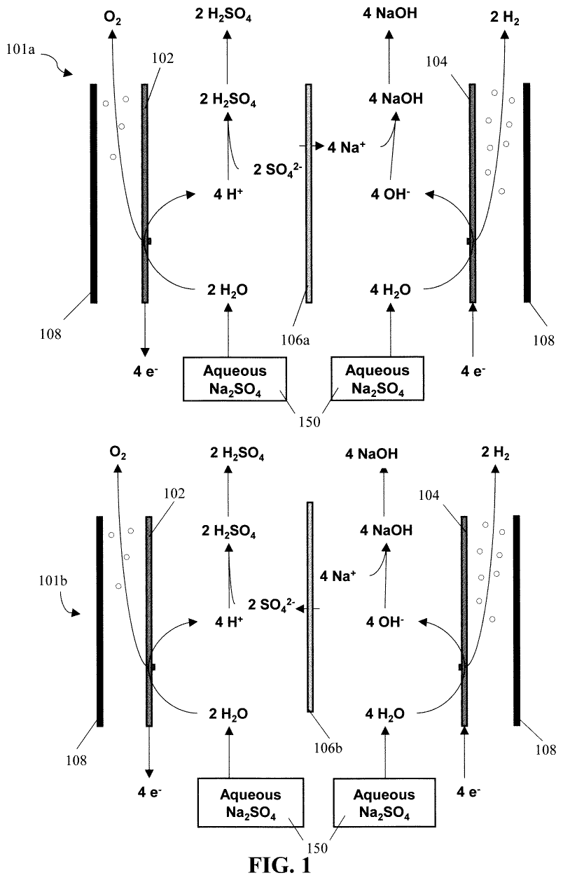

Turning now to the drawings, FIG. 1 provides a schematic illustration of (A) an exploded view of electrolyzer cell 101a comprising a cation exchange membrane; and (B) an exploded view of electrolyzer cell 101b comprising an anion exchange membrane. Electrolyzer cell 101a comprises an anode 102 spaced from a cathode 104, with a cation exchange membrane 106a between anode 102 and cathode 104. Aqueous electrolyte 150 comprising sodium sulfate is contacted with anode 102 and cathode 104 while a voltage potential is applied across anode 102 and cathode 104. One equivalent of oxygen gas and four equivalents of hydrogen ions are electrochemically generated at anode 102, while two equivalents of hydrogen gas and four equivalents of hydroxide ions are electrochemically generated at cathode 104 by transfer of four electrons between anode 102 and cathode 104. This results in formation of two equivalents of sulfuric acid at anode 102 and four equivalents of sodium hydroxide at cathode 104. The overall charge balances in the solutions contacting anode 102 and cathode 104 are maintained by exchange of sodium cations and hydrogen ions at cation exchange membrane 106a. Electrolyzer cell 101b comprises an anode 102 spaced from a cathode 104, with an anion exchange membrane 106b between anode 102 and cathode 104. Aqueous electrolyte 150 comprising sodium sulfate is contacted with anode 102 and cathode 104 while a voltage potential is applied across anode 102 and cathode 104. One equivalent of oxygen gas and four equivalents of hydrogen ions are electrochemically generated at anode 102, while two equivalents of hydrogen gas and four equivalents of hydroxide ions are electrochemically generated at cathode 104 by transfer of four electrons between anode 102 and cathode 104. This results in formation of two equivalents of sulfuric acid at anode 102 and four equivalents of sodium hydroxide at cathode 104, as in the case of electrolyzer cell 101a. The overall charge balances in the solutions contacting anode 102 and cathode 104 are maintained by exchange of sulfate anions and hydroxide ions at anion exchange membrane 106b.

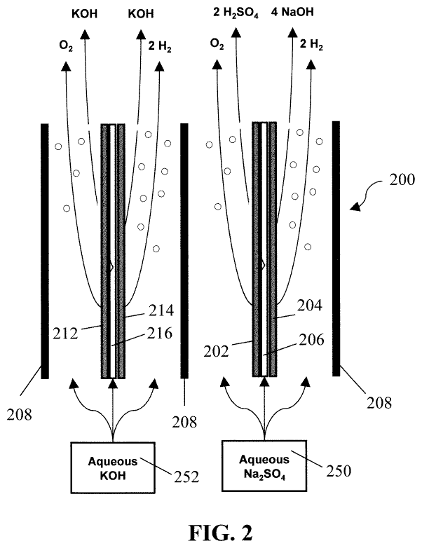

FIG. 2 provides a schematic illustration of a partially exploded view of an electrochemical E/AWE reactor unit 200 comprising an electrolyzer cell and an AWE cell. Reactor unit 200 comprises an electrolyzer cell portion and an AWE cell portion. The electrolyzer cell portion comprises anode 202 spaced from cathode 204, with an ion exchange membrane 206 (i.e., a CEM or an AEM) between anode 202 and cathode 204. The AWE portion comprises anode 212 spaced from cathode 214 with a porous diaphragm 216 between anode 212 and cathode 214. Reactor unit 200 is flanked by electrical insulators 208, with an additional electrical insulator 208 between the electrolyzer cell portion and the AWE cell portion. The spacing between the electrical insulators 208 and their nearest electrolyzer or AWE cell in FIG. 2 is exaggerated for ease of illustrating the various components. In use, first aqueous electrolyte 250 comprising sodium sulfate is contacted with anode 202 and cathode 204 while a voltage potential is applied across anode 202 and cathode 204. One equivalent of oxygen gas and four equivalents of hydrogen ions are electrochemically generated at anode 202, while two equivalents of hydrogen gas and four equivalents of hydroxide ions are electrochemically generated at cathode 204 by transfer of four electrons between anode 202 and cathode 204. This results in formation of two equivalents of sulfuric acid at anode 202 and four equivalents of sodium hydroxide at cathode 204. A second aqueous electrolyte 252 comprising potassium hydroxide is contacted with anode 212 and cathode 214 while a voltage potential is applied across anode 212 and cathode 214. One equivalent of oxygen gas is electrochemically generated at anode 212, while two equivalents of hydrogen gas are electrochemically generated at cathode 214 by transfer of four electrons, as in the case of the electrolyzer cell.

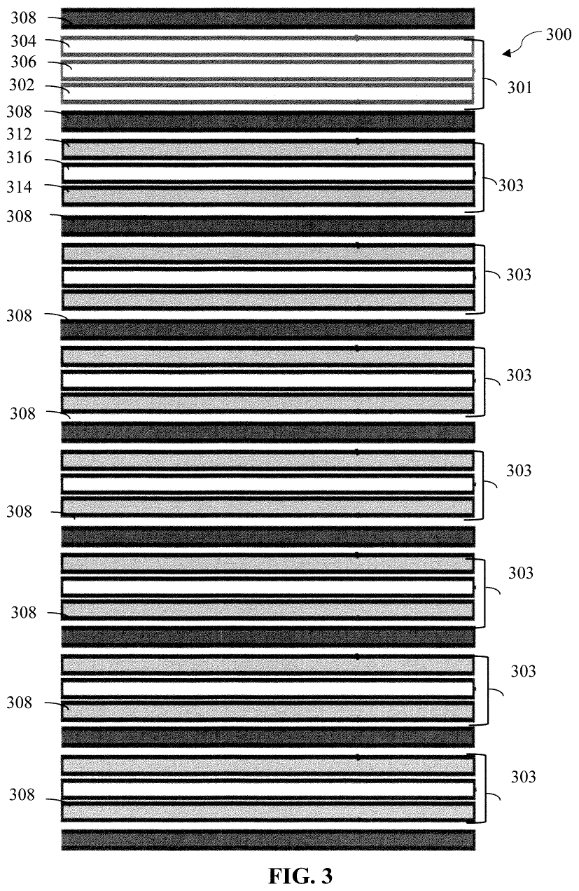

FIG. 3 provides a schematic illustration of an electrochemical E/AWE reactor unit 300 comprising an electrolyzer cell and seven AWE cells. Reactor unit 300 comprises an electrolyzer cell 301 stacked with seven AWE cells 303 each or which is separated from its nearest neighboring cell by an insulator 308, with additional electrical insulators 308 at each end of the stack. Electrolyzer cell 301 comprises anode 302 spaced from cathode 304, with an ion exchange membrane 306 (i.e., a CEM or an AEM) between anode 302 and cathode 304. Each AWE cell 303 comprises anode 312 spaced from cathode 314 with a porous diaphragm 316 between anode 312 and cathode 314. Reactor unit 300 operates in substantially the same way as reactor unit 200 of FIG. 2, except that each AWE cell 303 produces two equivalents of hydrogen, thereby resulting in a total output of 16 equivalents of hydrogen (2 from the electrolyzer cell 301, and 14 from the seven AWE cells 303) for each 4 electrons transferred between each anode and cathode pair in the reactor unit.

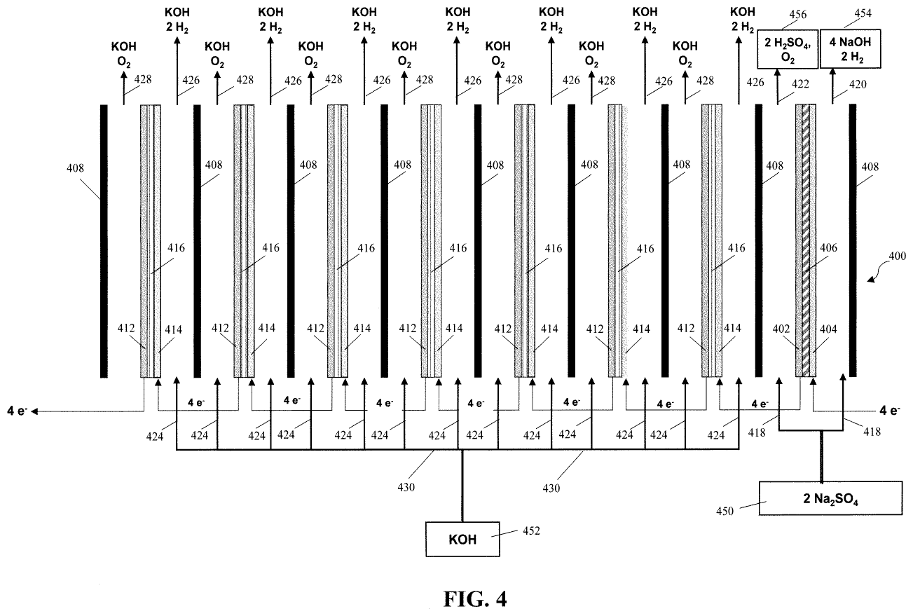

FIG. 4 provides a schematic illustration of a partially exploded view of an electrochemical E/AWE reactor unit 400 comprising an electrolyzer cell in a stack with seven AWE cells, in use to generate hydrogen, an acidic effluent comprising sulfuring acid, and an alkaline effluent comprising sodium hydroxide. The electrolyzer cell comprises anode 402 and cathode 404, with ion exchange membrane 406 (i.e., a CEM or an AEM) between anode 402 and cathode 404. Each AWE cell comprises anode 412 and cathode 414 with porous diaphragm 416 between anode 412 and cathode 414. The electrolyzer cell is electrically insulated from its nearest AWE cell by electrical insulator 408, each AWE cell is electrically insulated from its nearest AWE cells an additional electrical insulator 408, while the entire stack of electrolyzer and AWE cells is also flanked by electrical insulators 408.

In use, first electrolyte 450 comprising aqueous sodium sulfate is pumped through inlets 418 to contact anode 402 and cathode 404, while an electrical potential is applied across anode 402 and cathode 404. Alkaline effluent 454 comprising aqueous sodium hydroxide and hydrogen gas exits the electrolyzer cell through outlet 420, and acidic effluent 456 comprising aqueous sulfuric acid and oxygen exits the electrolyzer cell through outlet 422. At the same time, an electrical potential is applied across the anode 412 and the cathode 414 of each AWE cell while a second electrolyte comprising 20 to 40 wt % aqueous potassium hydroxide is pumped through manifold 430 into inlets 424 of each AWE cell to contact anodes 412 and cathodes 414. Hydrogen generated at each cathode 414 exits the AWE cells through outlets 426 along with the second electrolyte, and oxygen generated at each anode 412 exits the AWE cells through outlets 428 along with the second electrolyte.

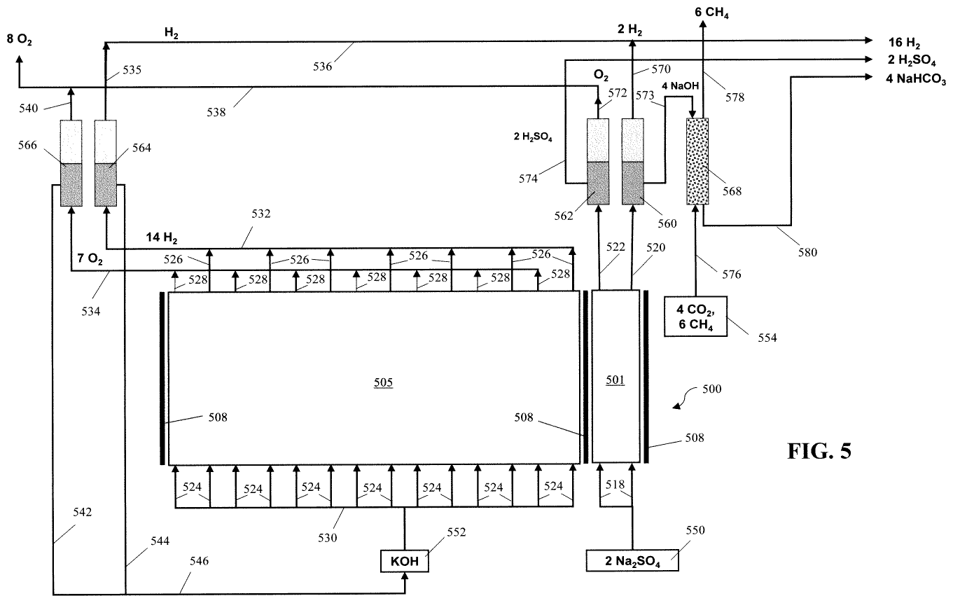

FIG. 5 provides a schematic illustration of an electrochemical E/AWE reactor unit comprising an electrolyzer cell and seven AWE cells integrated into a system for isolating the hydrogen produced by the reactor, isolating the acidic effluent, and generating bicarbonate from biogas contacted with the alkaline effluent. Reactor unit 500 is of substantially the same design as reactor unit 300 of FIG. 3 and reactor unit 400 of FIG. 4, and comprises electrolyzer cell 501 and a stack 505 of seven electrically insulated AWE cells (not shown to simplify illustration of other portions of the apparatus). Electrolyzer cell 501 is electrically insulated from the stack 505 of AWE cells by a first electrical insulator 508, and two additional electrical insulators 508 flank reactor unit 500. Each AWE cell within stack 505 is also separated from its nearest neighboring AWE cell by an electrical insulator (not shown) as in reactor units 300 and 400 of FIGS. 3 and 4, respectively. First electrolyte 550 (aqueous sodium sulfate) is pumped into electrolyzer cell 501 through inlets 518 to contact the anode and cathode of the electrolyzer cell (not shown). An alkaline effluent comprising aqueous sodium hydroxide and hydrogen gas exits electrolyzer cell 501 through outlet 520, and is channeled into gas/liquid separator 560 where hydrogen gas is separated from the aqueous sodium hydroxide liquid phase.

The separated hydrogen gas is channeled through outlet 570 into hydrogen collection line 536, while the liquid phase comprising aqueous sodium hydroxide is channeled through transfer line 573 into gas/liquid contactor 568, to contact biogas 554, which is pumped into contactor 568 through inlet 576. Gas/liquid contactor 568 is a porous material designed to facilitate mixing of the biogas with the aqueous sodium hydroxide. Carbon dioxide in biogas 554 reacts with the aqueous sodium hydroxide to form an aqueous sodium bicarbonate solution, and purified methane from the biogas is vented from contactor 568 through outlet 578 to be collected for use as a fuel. The aqueous sodium bicarbonate solution exits contactor 568 through outlet 580 to be collected for later use. An acidic effluent stream comprising oxygen gas and aqueous sulfuric acid exits electrolyzer cell 501 through outlet 522 after first electrolyte 550 contacts the anode of electrolyzer cell 501. The acidic effluent stream and oxygen gas enter gas/liquid separator 562, where the oxygen gas is separated from the aqueous sulfuric acid phase and is channeled into oxygen collection line 538 through outlet 572, while the aqueous sulfuric acid phase exits gas/liquid separator 562 through outlet 574 to be collected for later use.

At the same time, second electrolyte 552 comprising 20 to 40 wt % potassium hydroxide is pumped through manifold 530 into inlets 524 of each AWE cell of the stack 505 to contact the anodes and cathodes of the AWE cells in stack 505. Gaseous hydrogen generated at the AWE cathodes exist the AWE cells of stack 505 through outlets 526 along with second electrolyte 552, while oxygen gas generated at the AWE cell anodes exits the AWE cells of stack 505 through outlets 528 along with second electrolyte 552. The hydrogen gas generated at each AWE cathode is combined in manifold 532 connected to each outlet 526. Oxygen gas generated at each AWE cell anode is combined in manifold 534 connected to each outlet 528. Manifold 532 is in fluid communication with gas/liquid separator 564, which separates the hydrogen gas from second electrolyte 552 and channels the hydrogen through outlet 535 to hydrogen collection line 536. The separated second electrolyte 552 from separator 564 is channeled through recycle line 544 into manifold 546 to replenish electrolyte 552 being pumped into manifold 530. Manifold 534 is in fluid communication with gas/liquid separator 566, which separates the oxygen gas from second electrolyte 552 and channels the oxygen through outlet 540 to oxygen collection line 538. The separated second electrolyte 552 from separator 566 is channeled through recycle line 542 into manifold 546 to replenish electrolyte 552 being pumped in manifold 530. The hydrogen, aqueous sulfuric acid, and aqueous sodium bicarbonate are collected for later use, e.g., as feedstocks for biomethanation.

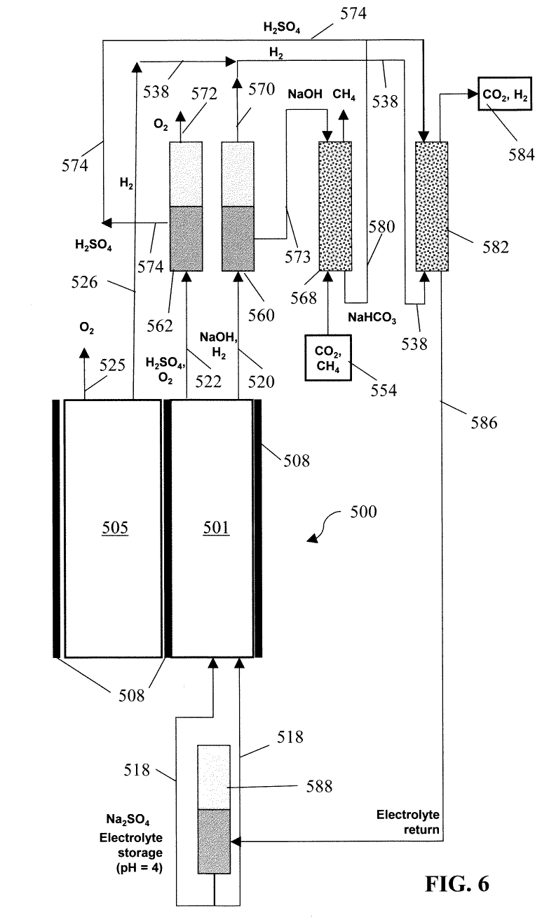

FIG. 6 provides a partial schematic illustration of the electrochemical reactor unit of FIG. 5 further illustrating conversion of the bicarbonate back to carbon dioxide gas and simultaneously generating an aqueous sodium sulfate solution, which is recycled as the first electrolyte for the reactor. In FIG. 6, the aqueous sodium bicarbonate exiting gas/liquid contactor 568 is combined with the aqueous sulfuric acid from outlet line 574 to neutralize the acid to form sodium sulfate and generate carbon dioxide gas. The mixture of aqueous sodium sulfate and carbon dioxide gas is channeled into a second gas/liquid contactor 582 along with the hydrogen from hydrogen collection line 538, where the hydrogen gas sweeps the carbon dioxide gas out of the gas/liquid contactor 582 resulting in a mixture of hydrogen and carbon dioxide that can be used as a feedstock for biomethanation. The aqueous sodium sulfate exits gas/liquid contactor 582 through outlet 586, and enters gas/liquid separator 588 to strip off any remaining gases (e.g., hydrogen or carbon dioxide). The resulting gas-depleted aqueous sodium sulfate is channeled into inlets 518 of electrolyzer 501 for use as the first electrolyte 550.

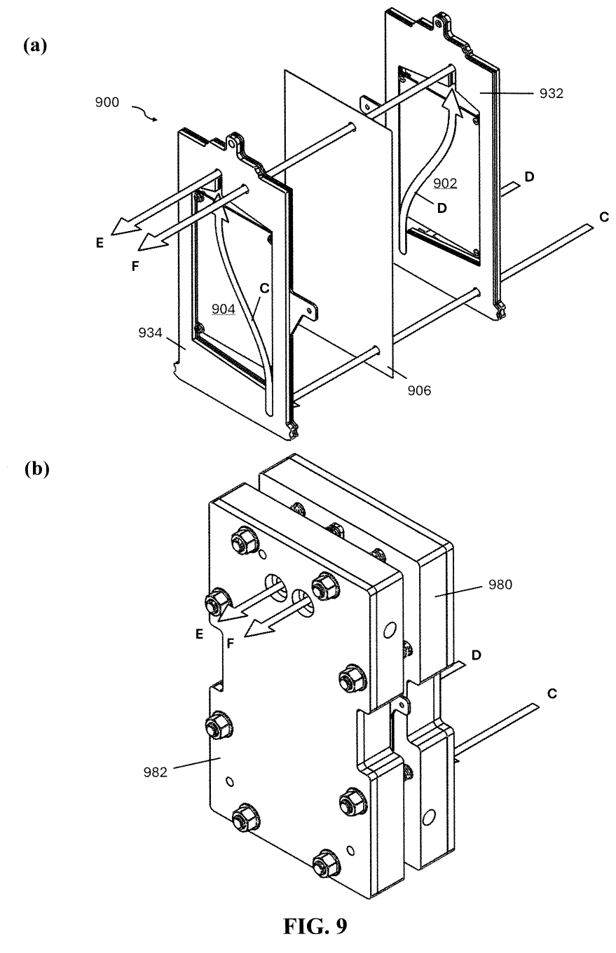

FIG. 9, panel (a) illustrates an embodiment of an electrolyzer cell as described herein, also referred to herein as a neutral water electrolysis cell or “NWE” cell. NWE cell 900 comprises anode 902 mounted within frame 932. Cathode 904 is mounted within frame 934, and ion-exchange membrane 906 is positioned between anode 902 and cathode 904, such that anode 902 with frame 932 is separated from cathode 904 and frame 934, thereby defining a separate anode chamber 935 and a separate cathode chamber 937. Flow C of neutral electrolyte passes through cathode chamber 938 and is converted to flow E of basic catholyte during use. Similarly, flow D of neutral electrolyte passes through anode chamber 936 and is converted to flow F of acidic anolyte during use. FIG. 9, panel (b) illustrates housing members 980 and 982 enclosing anode chamber 935 and cathode chamber 937, respectively, to keep the anode chamber separate from the cathode chamber, each of which shares a common wall formed by the ion exchange membrane 906.

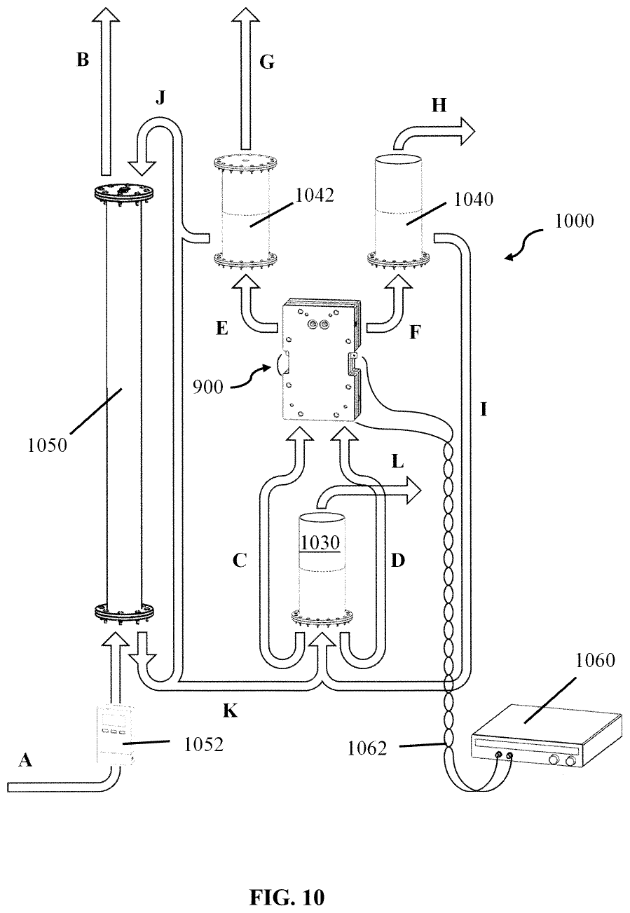

FIG. 10 schematically illustrates an NWE reactor system 1000 for producing, e.g., hydrogen and RNG. System 1000 comprises NWE cell 900 (as illustrated in FIG. 9) operably connected to electrolyte storage tank 1030 to supply flow D of neutral electrolyte to anode chamber 935 and flow C of neutral electrolyte to cathode chamber 937 of NWE cell 900 during use. When a voltage is applied across the anode and cathode via power source 1060 and wires 1062, neutral electrolyte from tank 1030 is pumped through the anode and cathode chambers of NWE 900. Water in the electrolyte is reduced in the cathode chamber to form hydrogen gas and hydroxide ion, which forms a basic catholyte that exits the cathode chamber (flow E) into a gas/liquid separator 1042 which separates and vents the hydrogen gas (flow G) and directs the liquid portion of the catholyte (flow J) into a gas/liquid contactor 1050. Flow A of feed gas (e.g., a biogas comprising methane and carbon dioxide) is fed into contactor 1050 via controller 1052 which flows through contactor 1050 in a direction opposite flow J feeding into contactor 1050. Hydroxide ion in flow J reacts with carbon dioxide from gas flow A to form bicarbonate and/or carbonate ion, thus stripping the carbon dioxide from flow A, leaving purified methane (e.g., RNG) to exit contactor 1050 via flow B. Flow K of basic catholyte and bicarbonate/carbonate ion flows into electrolyte storage tank 1030. Some carbon dioxide also vents from tank 1030 via flow L.

Simultaneously, water in the neutral electrolyte is oxidized in the anode chamber to form oxygen gas and hydrogen ion (H⁺), which forms an acidic anolyte that exits the anode chamber (flow F) into a gas/liquid separator 1040, which separates and vents the oxygen gas and carbon dioxide (flow H) and directs the liquid portion of the anolyte (flow I) into electrolyte storage tank 1030, where the neutral electrolyte is regenerated by reaction with the catholyte flow K, and carbon dioxide is liberated by reaction with the acidic anolyte.

The following non-limiting examples are provided to illustrate certain embodiments, features, and/or advantages of the reactors, systems and methods described herein.

### Example 1. Evaluation of Electrochemical Performance of the E/AWE Reactor Described Herein Compared to an ECC/AWE Reactor

An E/AWE reactor unit consisting of an electrolyzer cell and an adjacent AWE cell was prepared as described herein. For comparison, an ECC/AWE electrochemical reactor consisting of an ECC cell (including one carbon dioxide capture chamber and one acid generation chamber) and an adjacent AWE cell was prepared as described in USPA '417. The E/AWE and ECC/AWE reactors were evaluated using a surrogate biogas feed (35% carbon dioxide and 65% methane) under an applied electric field. The electrolytes used were aqueous sodium chloride for the ECC cell portion od the ECC/AWE reactor, aqueous sodium sulfate for the electrolyzer cell portion of the E/AWE reactor, and aqueous potassium hydroxide for the AWE cells.

The pH performances of the two reactors at the cathodes and anodes were evaluated during operation of the reactor at a current density of 12.5 mA/cm², a sodium chloride concentration of 5 g/L for the ECC cell and a concentration of sodium sulfate of 15 wt % for the electrolyzer cell. The results were collected by measuring the pH of the aqueous samples taken from the electrolytes contacting the anodes and cathodes of the ECC and electrolyzer cells of the reactors using a portable pH meter (Fisher ACUMET AP 62 portable pH meter). The results indicated that the E/AWE cell produced essentially the same level of alkalinity for carbon dioxide capture as the ECC/AWE reactor.

Biogas composition versus time was evaluated for a biomethanation system comprising the E/AWE reactor compared to a similar system using the ECC/AWE reactor. The employed biogas had a starting composition of 65% methane and 35% carbon dioxide. The results were collected by measuring gas compositions using gas chromatography (GC, Shimadzu GC 2014 TCD-FID), in both the gas samples collected from the effluent of the carbon dioxide capture chamber of the ECC cell and the and the headspace of the gas/liquid contactor connected to the alkaline and hydrogen-depleted effluent of the E/AWE reactor. The results indicated that the carbon dioxide and methane purification efficiencies were essentially 100% (i.e., essentially complete separation) after 30 minutes of operation for the E/AWE cell, while the carbon dioxide and methane purification efficiencies were slightly lower for the ECC/AWE reactor (i.e., a few percent contamination of methane with carbon dioxide and vice versa were observed for the ECC/AWE reactor).

FIG. 7 provides a bar graph showing hydrogen production for the E/AWE reactor compared to hydrogen production from the AWE cell alone. The applied current density was 100 mA/cm² and the sodium sulfate concentration of the electrolyzer cell electrolyte was 20 wt %. The KOH concentration of the AWE cell electrolytes was 25 wt %. The results were collected by measuring the gas samples by GC analysis. The results in FIG. 7 show that both the E/AWE reactor and the AWE cell produced hydrogen at the theoretical value of 0.061 NL/min, within the error limits of the test.

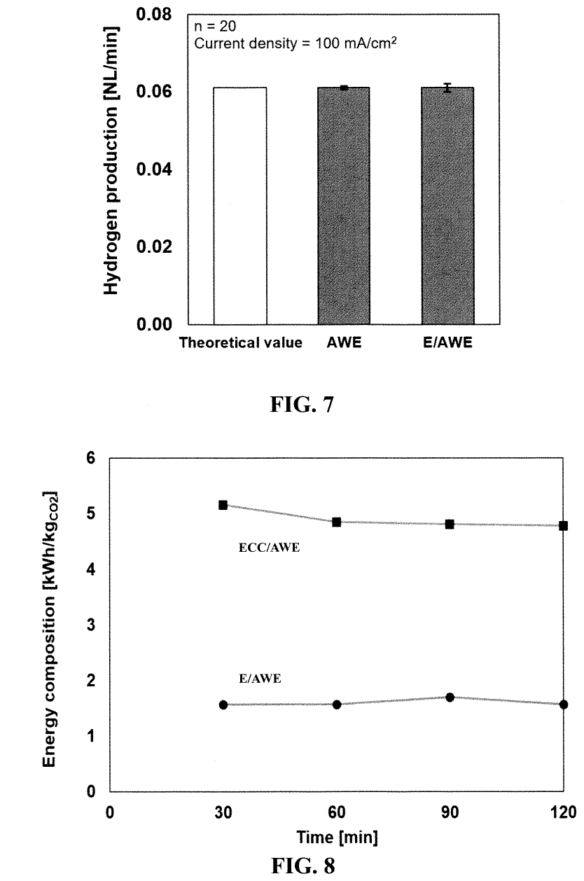

FIG. 8 provides plots of energy consumption of the E/AWE reactor compared to the ECC/AWE reactor over time. The results indicated that the E/AWE reactor consumed about 67% less energy, on average, compared to the ECC/AWE reactor under comparable operating conditions.

### Example 2. Comparison of Electrochemical Performance of an NWE Cell Compared to a Conventional AWE Cell

### Methods

### Detailed NWE and AWE Operation

AWE and NWE systems were operated with identical operation settings, but with different membranes and electrolytes (see Table 1). Both systems were run in electrolyte recirculation mode, while the NWE system was additionally operated in once-through mode to test its performance without residual inorganic carbon in the electrolyte. Five different biogas flow rates were tested for each system and current density, while each flow rate was maintained for one hour in once-through operation and for three hours in recirculation operation. During recirculation, the first two hours were used as a start-up phase to stabilize inorganic carbon concentrations in the electrolyte (as verified by inorganic carbon analysis). Gas composition and electrolyte pH were analyzed in the third hour.

### Characterization of System Performance

The specific energy consumption for CO₂ capture and release was calculated according to Eq. 3:

$${EC}_{CC} = \frac{I \cdot {U}_{{J}_{i}}}{{CR}_{{CO}_{2}} \cdot {\dot{m}}_{{CO}_{2 ( i n )}}} \qquad ( Eq . 3 )$$

where EC_{CC} is the energy consumption of CO₂ capture and release [kWh/kg_{CO}_{₂}], I is the current of the electrochemical cell [A], U_{J}_{ᵢ} is the voltage of the cell at a given current density Jᵢ [V], CR_{CO}_{₂} is the capture rate of CO₂ [%], and \dot{m}_{CO}_{₂}₍ᵢₙ₎ is the feed mass flow rate into the capture column [kg_{CO}_{₂}/h].

Ratios between molar flow rates of CO₂ and molar flow rates of electrons can be defined as current utilization (CU, Eq. 4):

$$Current utilization ( CU ) = \frac{{\dot{n}}_{{CO}_{2}}}{{\dot{n}}_{{e}^{-}}} [ \frac{\text{moles/d}}{\text{moles/d}} ] \qquad ( Eq . 4 )$$

The NWE cell was benchmarked against an AWE system regarding production rate and purity of H₂, while purity was also compared the ISO standard 14687:2019, requiring 98 vol % H₂. H₂ production rates were furthermore compared to their theoretical value (Eq. 5):

$${\dot{n}}_{{H}_{2}} = 3600 [ \frac{s}{hr} ] \cdot \frac{I}{F \cdot v} \qquad ( Eq . 5 )$$

where \dot{n}_{H2} is the specific H₂ molar production rate [mol/h], I is the applied current [A], F is the Faraday constant [=96,485.33 C/mol], and v is the valence constant [v=2 for H₂]. Equation 5 was furthermore used to calculate OH⁻ and H⁺ production but with a valence constant of 1.

The produced renewable natural gas (RNG) was evaluated against a minimum CH₄ purity of 95% according to DVGW technical rule G 260 [A]. The CO₂ capture rate of the NWE system was calculated according to Equation 6:

$${CR}_{{CO}_{2}} = \frac{{CO}_{2 ( i n )} - ( \frac{{CH}_{4 ( i n )}}{( 1 - {CO}_{2 ( out )} )} - {CH}_{4 ( i n )} )}{{CO}_{2 ( i n )}} \qquad ( Eq . 6 )$$

where CR_{CO2} is the capture rate of CO₂ [%], CO₂₍ᵢₙ₎ and CH₄₍ᵢₙ₎ are the feed gas concentrations [%], and CO₂₍ₒᵤₜ₎ is the outlet concentration [%]. In case of impurities in the feed and/or outlet gas, the above equation was normalized to CO₂ and CH₄ gas only.

TABLE 1
Operation of alkaline water electrolysis (AWE)
and neutral water electrolysis (NWE) cells.

| Parameter | Unit | AWE | NWE |
|---|---|---|---|
| Membrane | n/a | ZIRFON UTP | CMX-F6 |
| | | 500 diaphragm | cation exchange |
| | | | membrane |

Electrolyte

| | | | | |
|---|---|---|---|---|
| Composition | n/a | 25 wt % KOH sol. | 15 wt % Na₂SO₄ sol. | |
| Volume | [L] | 9 | 9 | |
| Temperature | [° C.] | 20 | 20 | |
| pH | n/a | >14 | 5.3 ± 0.4 | |
| Conductivity | [mS/cm] | 567* | 100.2 ± 0.6 | |
| Operation mode | n/a | Recirculation | Recirculation and | Recirculation and |
| | | | once-through | once-through |
| Current density | [mA/cm²] | 50, 100 | 50 | 100 |
| Biogas flow | [NL/d] | n/a | 50, 100, 150, | 100, 200, 300, |
| rates | | | 200, 250 | 400, 500 |
| \dot{n}_{co}_{₂}/\dot{n}ₑ_{^{−}} | [—] | n/a | 0.2, 0.4, 0.6, | 0.2, 0.4, 0.6, |
| | | | 0.8, 1.0 | 0.8, 1.0 |

Pump flow rates all [L/h]

| | | | |
|---|---|---|---|
| Electrolyte pumps | 6.3 | 6.3 | 6.3 |
| Catholyte supply pump | n/a | 6.3 | 6.3 |
| Catholyte | n/a | 35 | 35 |
| recirculation pump | | | |
| Agitation pumps | n/a | 360 | 360 |

*Literature value, as KOH conductivity exceeded the measuring range of the used conductivity meter.

### Analytical Methods

Product gas streams were analyzed for H₂, CH₄, CO₂, O₂, and N₂ using a gas chromatograph with a thermal conductivity detector (SRI 8610C MULTIPLE GAS ANALYZER #5, SRI Instruments, Torrance, CA, USA). Calibration of the gas chromatograph was conducted using different calibration gases (Gasco, Oldsmar, FL, USA), which were selected for expected component concentrations (i.e., 0.01%, 2%, 80%, and 99.99% for H₂, 0.01%, 65%, and 99.99% for CH₄, 0.04%, 35%, and 99.99% for CO₂, 0.25%, 5.12%, and 20.48% for O₂, and 1.7%, 20%, and 77.52% for N₂). Sampling bags were triple-flushed prior to usage with argon gas, which was also used as the gas chromatograph's carrier gas.

Inorganic carbon concentrations were analyzed using a total organic carbon analyzer (TOC-VCSN, Shimadzu, Kyoto, Japan). To prevent CO₂ outgassing from mildly alkaline or pH-neutral samples, sample pH was elevated using dilute KOH solution. A five-point calibration was performed with a 1,000 mg_{IC}/L stock solution containing 0.042 M of both NaHCO₃ and Na₂CO₃.

Measurement of pH was conducted using a benchtop PH meter (Accumet™ Basic AB315, Thermo Fisher Scientific Inc., Waltham, MA, USA). The meter was calibrated prior to each use using five buffer solutions traceable to the National Institute of Standards and Technology (NIST) standard, with nominal pH values of 1.68, 4.00, 6.86, 9.18, and 12.45.

The voltage of the electrochemical reactor between the cathodic and anodic current collectors was measured using a multimeter (Fluke 179 TRUE RMS MULTIMETER, Fluke Corporation, Everett, WA, USA).

The conductivity of the Na₂SO₄ electrolytes was measured using a handheld conductivity meter (SEVEN2GO, Mettler-Toledo, Schwerzenbach, Switzerland).

Theoretical pH values of electrolytes were calculated using the software PHREEQC (version 3.7.3, U.S. Geological Survey, Reston, VA, USA) using the Pitzer database for high ionic strength solutions.

Differences between data sets were assessed using heteroscedastic Student's t-tests, with p values of <0.05 and <0.01 indicating significant and highly significant differences, respectively.

### Design and Operation of the NWE Cell

The NWE cell was constructed similar to a conventional two-compartment AWE cell, comprising a nickel cathode (De Nora Tech, LLC., Concord, OH, USA), a dimensionally stable metal mesh (titanium) anode with an iridium-ruthenium electrocatalyst coating (The Electrode Supply, Redondo Beach, CA, USA), and a separating cation exchange membrane (CMX F6, Ameridia Innovative Solutions Inc., Napa, CA, USA). A 15 wt % Na₂SO₄ solution was used as working electrolyte and supplied to both anode and cathode compartments. During water electrolysis, the CEM enabled selective retention of SO₄⁻² and transfer of Na⁺ from anolyte to catholyte, thus balancing cathodic OH⁻ and anodic H⁺ production. As a result, the cathode compartment produced gaseous H₂ and an alkaline catholyte (containing Na₂SO₄ and NaOH), while the anode compartment produced gaseous O₂ and an acidic anolyte (containing Na₂SO₄ and H₂SO₄).

For realization to practice, electrodes (with a surface area of 87.1 cm² each) were mounted onto titanium current collectors with embedded flow channels in a near-zero gap design encased by two polyvinylchloride boards and stainless-steel endplates for a sturdy, leak-free assembly as shown in FIG. 9, panels (a) and (b).

The full experimental setup is shown in FIG. 10, which employed a peristaltic pump (not shown) to supply the Na₂SO₄ electrolyte from a 5 L electrolyte storage tank to the anode and cathode chambers of the electrochemical cell. The two gas/liquid streams subsequently exiting the cell were separately routed into 5 L gas/liquid separators, with the separator for the O₂/anolyte stream open to the atmosphere, and the headspace of the gas/liquid separator for the H₂/catholyte connected to a 6 mm outlet tubing for H₂ collection. Following H₂ separation, the catholyte was pumped to the top of a gas/liquid contactor column (filled with plastic pellet packing), where it was brought into contact with synthetic biogas (65%+1% CH₄, balance CO₂, Airgas Inc., Radnor, PA, USA) that was fed from the bottom of the column via a mass flow controller (SMART-TRAK 2 SERIES 100, Sierra Instruments, Monterey, CA, USA).

After CO₂ capture in the column, the upgraded biogas (i.e., RNG) was collected at the top using gas sampling bags, while the captured CO₂ was discharged from the bottom as aqueous HCO₃⁻/CO₃²⁻ together with the catholyte. To ensure proper wetting of the packing material, a side-stream recirculation loop continuously rerouted some of the catholyte back to the top of the column. To facilitate the subsequent release of the captured CO₂ and electrolyte regeneration, the catholyte was recombined with the acidic anolyte and agitated using a submersible aquarium pump in the electrolyte storage tank. A second aquarium pump was added to the O₂ gas/liquid separator to degas residual CO₂. In addition to the depicted recirculation operation, the setup was also operated in once-through operation in order to compare CO₂ capture in fresh vs. recirculated electrolyte.

Using a direct current power supply (AMETEK Programmable Power Inc., San Diego, CA, USA), the electrochemical cell was operated at two constant current densities (50 and 100 mA/cm²), while biogas was supplied at flow rates ranging from 50 to 500 NL/d (or 17.5-175 NL_{CO2}/d, corresponding to the 35% proportion of CO₂ in biogas). The resulting ratios between molar flow rates of CO₂ and molar flow rates of electrons ranged from 0.2 to 1.0 mole CO₂ per mole e and current utilization was calculated according Eq. 4 described above.

In addition to the above setup, a reference AWE cell was used (serving as a benchmark in terms of H₂ production), featuring the same cell configuration, except that the CEM was replaced by an industry-typical AGFA ZIRFON UTP 500 diaphragm (Agfa-Gevaert NV, Mortsel, Belgium) and that 25 wt % KOH solution was used instead of the 15 wt % Na₂SO₄ electrolyte. A summary table with further details on the operation of both NWE and AWE is presented in Table 1.

H₂ purity and H₂ Production Rates of NWE

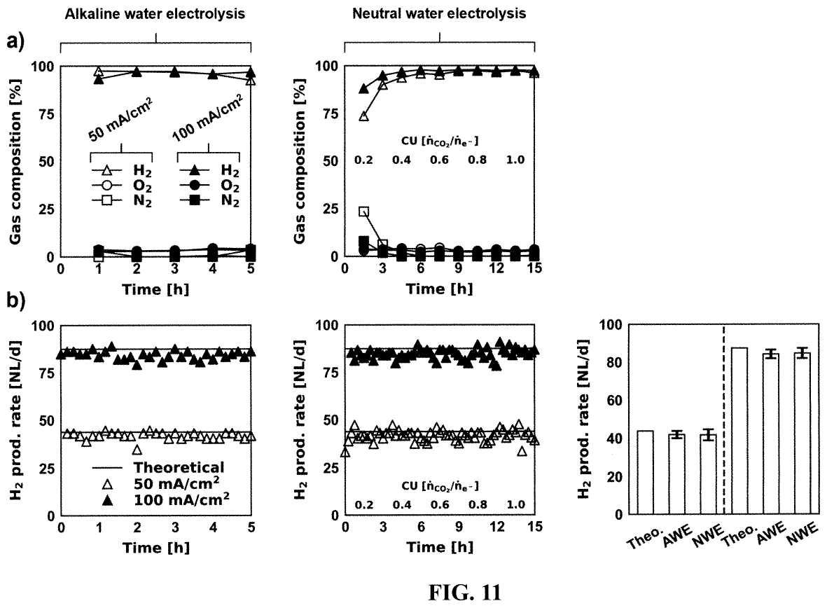

H₂ composition and production rates are displayed in FIG. 11 for both the AWE reference system and the NWE system. Both systems achieved high average H₂ purities of ˜97% (after displacing N₂ gas from initial headspace flushing), with the remaining ˜3% being O₂ (FIG. 11, panel (a)). Notably, NWE's H₂ product gas was entirely free from CH₄ and CO₂ despite the electrolyte's exposure to biogas and the resulting presence of inorganic carbon in the catholyte. This non-detection is credited to the low solubility of CH₄ (˜22 mg/L in water at 25° C.) with negligible carryover, and the basic pH of the catholyte (>11), which effectively prevented CO₂ outgassing. Accordingly, the difference between AWE and NWE in terms of H₂ purity was found to be statistically insignificant (according to a Student's t-test, at p >0.05), suggesting that biogas upgrading does not compromise H₂ product gas quality in the NWE system.

The relatively high O₂ concentration of ˜3% in both AWE and NWE was attributed to the recombination of catholyte and anolyte streams in the electrolyte storage container, as the anolyte carried non-buoyant O₂ microbubbles that were subsequently released in the cathode compartment. While such ˜3% O₂ impurity rendered the produced H₂ gas non-compliant with ISO requirements (>98% H₂, <1.9% O₂), simple mitigation strategies like filter meshes or electrolyte degassing can likely prevent such microbubble carryover. Catholyte/anolyte separation, on the other hand, which is a common strategy to minimize foreign gas intrusion in AWE systems, is not suitable for NWE systems, as mixing of the CO₂-rich catholyte with the acidic anolyte is a prerequisite for subsequent CO₂ degassing. Naturally, the observed impurity will also become more ancillary at higher (and more practically relevant) current densities, as higher gas production rates lead to a dilution of foreign gas intrusions. Data from this study supports this hypothesis, as O₂ concentrations were slightly but significantly lower for 100 mA/cm² (i.e., 2.47±0.26%) than for 50 mA/cm² (i.e., 3.31±0.64%) in the NWE system, according to a t-test at p<0.05.

H₂ production rates (FIG. 11, panel (c)) coincided well with their theoretical values for both current densities (with average differences of less than 3%), indicating a current efficiency of nearly 100% for the water electrolysis reaction. Differences in H₂ production rates between AWE and NWE were found to be statistically insignificant (at pₘᵢₙ=0.47), which can be attributed to the electrochemical stability of both electrolytes with no side reactions. Overall, results indicate that AWE and NWE can be used interchangeably for H₂ production and that adding CO₂ capture capability has no negative impact on achievable H₂ purity and current efficiency.

### Alkalinity and Acidity Production in the NWE System

In electrolyte once-through operation the NWE was able to successfully produce a strongly acidic anolyte (pH_{avg} 2.41±0.10) and an alkaline catholyte (pH_{avg} 12.33=0.14) from the employed Na₂SO₄ electrolyte. The measured pH values had maximum differences of 7.5% for the anolyte and 0.6% for the catholyte. The concordance between measured and theoretical values suggests high current efficiency of acidity/alkalinity generation, as calculation of the theoretical values assumed 100% current efficiency with production of 1 mole of NaOH and 0.5 moles H₂SO₄ per mole of e supplied. However, an exact quantification of current efficiency regarding acidity/alkalinity production remains somewhat difficult, due to the limited reliability of pH calculations at high ionic strengths.

Exposing the catholyteᵢₙ stream to biogas within the gas/liquid contactor column caused a drop in pH due to NaOH consumption towards NaHCO₃/Na₂CO₃ formation, while the pH reduction expectedly showed an increasing trend with increasing current utilization (CU) values. The preservation of relatively high pH values at CU=1.0 (e.g., pH=9.6 at 100 mA/cm²) points to somewhat incomplete CO₂ capture, which may be attributed to limited mass transfer in the employed gas/liquid contactor column, as well as reduced capture kinetics and unfavorable speciation at less basic pH levels. However, engineering optimizations of the contactor column (e.g., use of a wire gauze instead of plastic pellets, or different column design with reduced wall effects) are expected to effectively improve CO₂ capture, with a concurrent decrease in effluent pH.

When operated in recirculation mode, pH dynamics were further impacted by inorganic carbon accumulation in the electrolyte. Inorganic carbon concentrations initially increased with every new CU (or biogas flow rate) but reached a plateau after approximately 1.5-2.0 hours. While such trend indicated initially incomplete degassing of the captured CO₂ (despite the use of agitation pumps, the acidity in both electrolyte storage tank and O₂ gas/liquid separator, and the CO₂ stripping effect of the O₂ evolution reaction), the stabilization of inorganic carbon concentrations after ˜₂ hours suggests attainment of equal CO₂ capture and release rates at an increased inorganic carbon baseline.

Catholyteᵢₙ PH dropped as a function of electrolyte inorganic carbon concentrations due to the increased inorganic concentrations and the corresponding electrolyte buffering, which translated to a slight but system-wide reduction in pH compared to once-through operation. Since anolyte and catholyte nonetheless retained strongly acidic and alkaline levels, respectively, results suggest that NWE's CO₂ capture and release capability was preserved even in recirculation mode.

### Co₂ Capture Rates and RNG Quality

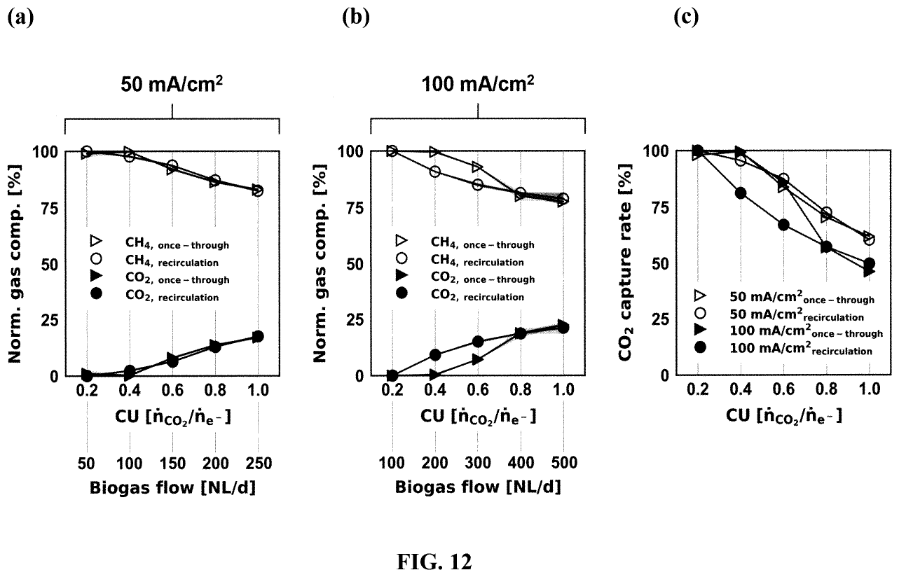

The NWE system achieved near-complete CO₂ capture (>98%) and high-purity CH₄ concentrations of ≥99% at the two lowest CUs (i.e., 0.2 and 0.4) at both current densities in once-through operation (FIG. 12, panels (a) and (b)). A similar observation was made for recirculation operation, albeit with a first departure from near-complete CO₂ capture at CU=0.4 and a current density of 100 mA/cm², with a resulting CH₄ purity of 90.8% (FIG. 12, panel (a)). Further reductions in CO₂ capture rates and RNG qualities occurred at CUs ≥0.6 (FIG. 4c), due to shortened residence times in the gas/liquid contactor column and the slowing CO₂ capture kinetics with decreasing catholyte alkalinity. The overall lowest capture rates and CH₄ purities were observed in recirculation mode at CU=1.0 and 100 mA/cm² and were 50% and 79%, respectively. Notwithstanding, results suggest that the novel NWE system is capable of purifying biogas to DVGW G 260 Technical Rule RNG standards of >95% CH₄.

Considering H₂ production as well, the NWE cell was able to concurrently produce a total of 88 NL/d H₂ and 130 NL/d high-purity RNG with CH₄ >99% at CU=0.4 and 100 mA/cm² in once-through operation, thus demonstrating its role as a two-fold power-to-gas system. The maximum observed gas production that was achieved amounted to 88 NL/d H₂ and ˜413 NL/d RNG, albeit with a lower CH₄ purity of 79% (at CU=1.0 and 100 mA/cm²). When normalizing the above production rates to an electrode/membrane area of 1 m², results correspond to ˜10 m³/d H₂ as well as 15 m³/d RNG (at >99% CH₄) or 47 m³/d RNG (at 79% CH₄).

The slightly higher capture rates in once-through operation relative to recirculation operation stem from the more basic pH of the fresh electrolyte without residual inorganic carbon. However, it is noteworthy that the difference between the two operation modes was almost negligible at 50 mA/cm², and only significant for CUs of 0.4 and 0.6 at 100 mA/cm². This result indicates that operation in recirculation mode is feasible with only slightly diminished performance, but with the key advantage of avoiding continuous Na₂SO₄ demand and saline waste brine generation.

Experimental results further suggest the purification capability of the NWE system increases nearly linearly with increasing current density. Influent biogas flow rates at 100 mA/cm² were twice as high as at 50 mA/cm², while yielding only slightly inferior CH₄ purities. Since current densities in this study were only limited by the maximum allowable power of the employed power supply, not by the materials used, further increases in system capacity at higher current seem attainable. This also marks a distinct advantage of NWE over competing electrochemical CO₂ capture systems (e.g., bipolar membrane electrodialysis), as those systems are typically limited to current densities of ˜50 mA/cm² due to the delamination risk of bipolar membranes.

### Energy Consumption of NWE Cell

Cell voltages and power usages of both AWE and NWE systems were assessed. For the AWE system, voltages amounted to 1.77 V for 50 mA/cm² and 1.91 V for 100 mA/cm² (hereby showing excellent agreement with literature values on room-temperature AWE), while the NWE system reached substantially higher cell voltages of 2.78 V at 50 mA/cm² and 3.09 V at 100 mA/cm². The offset is mainly attributed to the electrolytic pH shift in the NWE cell, which causes unfavorable kinetics at both anode and cathode and is mathematically described as the added Nernst potential of Φ=0.059 ΔpH [V]. When assuming an extreme pH gradient of 14 between anode and cathode surfaces, cell voltage increases by 0.83 V, thus theoretically elevating voltage drops from 1.77 (AWE) to 2.60 V (NWE) at 50 mA/cm² and from 1.91 V (AWE) to 2.74 V (NWE) at 100 mA/cm². (The assumption of a pH gradient larger than the difference between measured average anolyte and catholyte pH levels [i.e., ˜10] seems justified, as pH measurements reflect the bulk electrolyte and not the boundary layers at the electrodes.) A second contributor to the difference in cell voltage is the lower ionic conductivity of 15 wt % Na₂SO₄ electrolyte (˜100 mS/cm) relative to 25 wt % KOH (˜570 mS/cm). However, to precisely quantify the individual contributions of electrolytic pH shift and ionic conductivity on the increased NWE cell voltage, further modeling studies or pH measurements directly at the electrodes are necessary.

Interestingly, NWE cell voltage was found to increase slightly over time during recirculation operation. While the cell voltage largely stabilized during the initial three hours of the experiment (out of a total of 15 hours), it nonetheless kept rising at a rate of ˜6 mV/h towards the end of the experiment. Possible causes are a slight decline in electrolyte conductivity with increasing inorganic carbon concentrations, and/or an early onset of anode degradation in the extremely acidic and oxidizing anolyte environment. To prevent an underestimation of energy demand, the highest observed cell voltage was therefore used to calculate NWE's power usage and energy demand. Further long-term testing is necessary to verify electrode stability over extended periods of time.

The electric power usage of both AWE and NWE systems was calculated by adding the product of cell voltage and applied current to the pump power usage. Notably, the power usages of the electrolyte pumps were almost negligible for AWE operation (˜0.05 W), while the two agitation pumps employed in the NWE setup consumed an extra 3 W, corresponding to an increase in power usage by +24% and +10% for 50 mA/cm² and 100 mA/cm², respectively.

The resulting specific energy consumptions for CO₂ capture at different CUs were evaluated. The results indicated that energy consumptions for CO₂ capture varied predominately as a function of CU, spanning from 3.5 kWh/kg_{CO2} to 10 kWh/kg_{CO2} (when disregarding H₂ production) and from 1.0 kWh/kg_{CO2} to 3.5 kWh/kg_{CO2} (when considering only the added energy consumption for CO₂ capture relative to AWE). Energy consumption decreased as CUs increased, and accordingly, the minimum added energy consumption was identified at CU=1.0 (at 50 mA/cm²) and amounted to 1 kWh/kg_{CO2}, albeit with a corresponding RNG purity of only 83% CH₄. The lowest added energy consumption that still maintained target RNG quality (i.e., >95% CH₄)⁴ amounted to 1.6 kWh/kg_{CO2} at 50 mA/cm² and to 3.6 kWh/kg_{CO2} at 100 mA/cm². Thus, applications with less stringent (or no) effluent gas requirements (e.g., CO₂ capture from flue gas, direct air capture) would show even greater promise for this NWE approach, with projected additional energy consumptions of well below 1 kWh/kg_{CO2}. Improved energy efficiency of the CO₂ degassing process (now conducted using simple aquarium pumps) as well as better mass transfer in the gas/liquid contact column are further examples on how to lower CO₂ capture energy consumption.

When compared to other electrochemical CO₂ capture technologies in the literature, like bipolar membrane electrodialysis and electrochemical CO₂ adsorbent regeneration the NWE system described herein falls well within commonly reported ranges of energy consumption; relative to previous NWE studies, while offering the key advantages of a simple cell design with the same footprint as an AWE cell and an eliminating need for current-limiting, and easy-to-deteriorate bipolar membranes.

### Discussion

NWE was established as a novel power-to-gas process for green H₂ production and concurrent biogas upgrading to RNG by leveraging the inherent acidity/alkalinity production of water electrolysis. Key advantages of the process include the simple cell design, the exclusive use of commercially available materials, its operation without chemical waste, and a competitive energy consumption for carbon capture.

Maximum CO₂ capture rates and resulting CH₄ purities approached nearly 100%, while H₂ production rates and purities were found to be interchangeable with an AWE reference system. Added energy consumptions for carbon capture ranged from 1.0 to 3.6 kWh/kg_{CO2}, which compared well to other electrochemical CO₂ capture approaches, but resembled the lowest reported energy consumption for NWE systems. The highest energy-efficiencies (but also the lowest CH₄ purities) were obtained at the highest biogas flow rates, indicating that NWE might show even higher efficiency for carbon capture applications with low or no product gas requirements (e.g., flue gas treatment, direct air capture).

Further measures to decrease energy consumption for NWE carbon capture include common strategies for enhanced AWE efficiency, such as higher operating temperature for improved ionic conductivity, or elevated pressure for decreased bubble size and ohmic resistance in the electrolyte. Besides, use of porous electrodes with maximized surface area and electrocatalysts specifically designed for pH environments of NWE cells might further contribute to lowering overpotentials and energy consumption. Improvements of the capture/release performance of the employed gas/liquid contactor column are further contributors to higher efficiency and may include increased contact surface or the addition of CO₂-to-HCO₃ catalysts. Electrolyte engineering towards optimized ionic conductivity, baseline pH, CO₂ capture/release kinetics inside or outside the NWE cell, and total CO₂ capture capacity represent another important avenue for improved energy-efficiency.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The embodiments of the invention in which an exclusive property or privilege is claimed are defined as follows:

1. An electrochemical reactor useful for capturing carbon dioxide and generating hydrogen; the reactor comprising: at least one reactor unit including an electrolyzer cell and a first alkaline water electrolysis (AWE) cell electrically insulated from the electrolyzer cell, the reactor unit being flanked by electrical insulators;
wherein the electrolyzer cell comprises:
(a) a first anode;
(b) a first cathode; and
(c) an ion exchange membrane between the first anode and the first cathode; wherein the ion exchange membrane is selected from the group consisting of a cation exchange membrane and an anion exchange membrane; and
the first AWE cell comprises:
(d) a second anode;
(e) a second cathode; and
(f) a porous diaphragm between the second anode and the second cathode.

2. The electrochemical reactor of claim 1, further comprising:
(i) a first inlet adapted and arranged to allow a flow of a first liquid electrolyte to contact the first anode during use;
(ii) a first outlet positioned opposite the first inlet in fluid communication with the first inlet;
(iii) a second inlet adapted and arranged to allow a flow of the first liquid electrolyte to contact first cathode during use;
(iv) a second outlet positioned opposite the second inlet in fluid communication with second inlet;
(v) a third inlet adapted and arranged to allow a flow of a second liquid electrolyte to contact the second anode during use;
(vi) a third outlet positioned opposite the third inlet in fluid communication with the third inlet;
(vii) a fourth inlet adapted and arranged to allow a flow of the second liquid electrolyte to contact the second cathode during use; and
(viii) a fourth outlet positioned opposite the fourth inlet in fluid communication with the fourth inlet.

3. The electrochemical reactor of claim 1, wherein the electrochemical reactor comprises a stack of 2 to about 50 reactor units.

4. The electrochemical reactor of claim 2, wherein the electrochemical reactor comprises a stack of 2 to about 50 reactor units.

5. The electrochemical reactor claim 1, wherein each reactor unit comprises 1 to 10 additional AWE cells stacked with the first AWE cell, and each additional AWE cell was electrically insulated from the first AWE cell and the other additional AWE cells.

6. The electrochemical reactor claim 2, wherein each reactor unit comprises 1 to 10 additional AWE cells stacked with the first AWE cell, and each additional AWE cell was electrically insulated from the first AWE cell and the other additional AWE cells.

7. The electrochemical reactor of claim 6, further comprising:
a first manifold connecting the third and fourth inlets of each AWE cell to distribute the flow of the second electrolyte to the second anode and second cathode during use;
a second manifold connecting the third outlets of each AWE cell to combine the flow of the second electrolyte exiting each third outlet during use; and
a third manifold connecting the fourth outlets of each AWE cell to combine the flow of the second electrolyte exiting each fourth outlet during use.

8. The electrochemical reactor of claim 1, wherein the first and second cathodes and the first and second anodes comprise non-porous conductive plates.

9. The electrochemical reactor of claim 8, wherein the non-porous conductive plates comprise a material selected from the group consisting of nickel, titanium, stainless steel, and a Magnéli phase titanium oxide.

10. The electrochemical reactor of claim 1, wherein the first and second cathodes and the first and second anodes comprise porous conductive plates.

11. The electrochemical reactor of claim 10, wherein the porous conductive plates are porous plates of a material selected from the group consisting of nickel, titanium, stainless steel, and a Magnéli phase titanium oxide.

12. A method for simultaneously electrochemically generating hydrogen, oxygen, an acidic solution, and a bicarbonate solution comprising the steps of:
(a) providing the electrochemical reactor of claim 1;
(b) contacting a flow of a first liquid electrolyte comprising an aqueous solution of a neutral salt with the first anode and the first cathode of the electrolyzer cell of the reactor;
(c) contacting a flow of a second liquid electrolyte comprising an aqueous alkaline solution with second anode and the second cathode of the AWE cell of the reactor;
(d) placing the first anode and the first cathode of the electrolyzer cell in circuit with a DC power source; and
(e) placing the second anode and the second cathode of the AWE cell in circuit with a DC power source;
wherein hydrogen gas and hydroxide ions are electrochemically generated at the first cathode within the flow of the first liquid electrolyte contacting the first cathode, and the flow of the first liquid electrolyte becomes an alkaline first effluent containing hydrogen gas after contact with the first cathode ceases; oxygen gas and hydrogen ions are electrochemically generated at the first anode within the flow of the first liquid electrolyte contacting the first anode, and the flow of the first liquid electrolyte contacting the first anode becomes an acidic second effluent containing oxygen after contact with the first anode ceases; hydrogen gas and hydroxide ions are electrochemically generated at the second cathode within the flow of the second liquid electrolyte contacting the second cathode, and the flow of the second liquid electrolyte becomes an alkaline third effluent containing hydrogen gas after contact with the second cathode ceases; oxygen gas and hydrogen ions are electrochemically generated at the second anode within the flow of the second liquid electrolyte contacting the second anode, and the flow of the second liquid electrolyte contacting the second anode becomes an alkaline fourth effluent containing oxygen after contact with the first anode ceases;
(f) separating and collecting the hydrogen gas from the first effluent to afford a hydrogen-depleted alkaline first effluent;
(g) separating the oxygen gas from the second effluent to afford an oxygen-depleted acidic second effluent;
(h) separating and collecting the hydrogen gas from the third effluent to afford a hydrogen-depleted alkaline third effluent;
(i) separating the oxygen gas from the fourth effluent to afford an oxygen-depleted alkaline fourth effluent;
(j) contacting the hydrogen-depleted alkaline first effluent with a carbon dioxide-rich gas to generate a bicarbonate containing solution by reaction of hydroxide ions in the hydrogen-depleted alkaline first effluent with carbon dioxide;
(k) separating and collecting methane from the bicarbonate-containing solution.

13. The method of claim 12, wherein the first liquid electrolyte comprises aqueous sodium sulfate.

14. The method of claim 13, wherein the sodium sulfate is present in the first liquid electrolyte at a concentration of about 5 wt % to about 30 wt %.

15. The method of claim 12, wherein the second liquid electrolyte comprises aqueous sodium hydroxide or aqueous potassium hydroxide.

16. The method of claim 15, wherein the sodium hydroxide or the potassium hydroxide is present in the second liquid electrolyte at a concentration of about 20 wt % to about 40 wt %.

17. The method of claim 12, further comprising the steps of:
(A) combining the hydrogen collected in steps (f) and (h);
(B) contacting the hydrogen-depleted alkaline second effluent with the hydrogen-depleted acidic first effluent to generate carbon dioxide gas and a neutral salt solution; and
(C) separating and collecting the carbon dioxide gas generated in step (B).

18. The method of claim 17, further comprising the steps of:
(D) adding the hydrogen from step (A) and the carbon dioxide gas from step (C) to a biomethanation reactor charged with microorganisms for converting hydrogen and carbon dioxide to methane; and
(E) collecting methane generated by the microorganisms.

19. The method of claim 18, further comprising combining the methane collected in step (k) with the methane collected in step (E).

20. A neutral water electrolysis reactor comprising:
(1) an electrolyzer cell comprising an anode; a cathode; and an ion exchange membrane between the anode and the cathode, separating the electrolyzer cell into an anode chamber and a cathode chamber; wherein the ion exchange membrane is selected from the group consisting of a cation exchange membrane and an anion exchange membrane;
(2) an electrolyte storage tank for supplying an aqueous neutral electrolyte to the electrolyzer cell;
(3) the anode chamber and the cathode chamber being adapted and arranged in fluid communication with the electrolyte storage tank to allow separate flows of the aqueous neutral electrolyte to contact the anode and the cathode, respectively, during use;
(4) a first gas/liquid separator in fluid communication with the anode chamber; the first gas/liquid separator being adapted and arranged to separate gases from an acidic anolyte discharging from the anode chamber during use;
(5) a second gas/liquid separator in fluid communication with the cathode chamber; the second gas/liquid separator being adapted and arranged to separate gases from a basic catholyte discharging from the cathode chamber during use;
(6) the electrolyte storage tank being in fluid communication with the first gas/liquid separator such that a liquid portion of the acidic anolyte discharging from the anode chamber during use is directed into the electrolyte storage tank; and
(7) a porous gas/liquid contactor in fluid communication with the second gas/liquid separator; the second gas/liquid separator being adapted and arranged to direct a flow of a liquid portion of the basic catholyte into the gas/liquid contactor to contact a counterflow of a gas stream comprising carbon dioxide and methane within the gas/liquid contactor during use; the porous gas/liquid contactor also being in fluid communication with the electrolyte storage tank such that basic catholyte containing carbonate and/or bicarbonate ion discharging from the contactor during use is directed into the electrolyte storage tank;
wherein in use, a voltage is applied across the anode and the cathode, and neutral electrolyte from the electrolyte storage tank is circulated through the electrolyzer cell; the gas stream comprising carbon dioxide and methane is introduced into the gas/liquid contactor; water in the neutral electrolyte circulating through the anode chamber is oxidized to form oxygen gas and hydrogen ion, thereby producing the acidic anolyte; water in the neutral electrolyte flowing through the cathode chamber is reduced to form hydrogen gas and hydroxide ion; thereby producing the basic catholyte; the acidic anolyte and basic catholyte containing carbonate and/or bicarbonate entering the electrolyte storage tank regenerate the neutral electrolyte within the tank; the hydrogen gas separated from the basic catholyte in the second gas/liquid separator is collected for later use; the basic catholyte flowing into the gas/liquid contactor reacts with gaseous carbon dioxide from the gas stream to sequester the carbon dioxide as carbonate and/or bicarbonate ions; and a carbon-dioxide-depleted methane-containing gas stream is vented from the gas/liquid contactor and is collected for later use.

21. The neutral water electrolysis reactor of claim 20, wherein the first and second cathodes and the first and second anodes comprise non-porous conductive plates.

22. The neutral water electrolysis reactor of claim 21, wherein the non-porous conductive plates comprise a material selected from the group consisting of nickel, titanium, stainless steel, and a Magnéli phase titanium oxide.

23. The neutral water electrolysis reactor of claim 20, wherein the first and second cathodes and the first and second anodes comprise porous conductive plates.

24. The neutral water electrolysis reactor of claim 23, wherein the porous conductive plates are porous plates of a material selected from the group consisting of nickel, titanium, stainless steel, and a Magnéli phase titanium oxide.

25. The neutral water electrolysis reactor of claim 20, wherein the ion-exchange membrane is a cation-exchange membrane.
